SEQUENCE LISTING (24 sequences)

SEQ ID NO: 1 - DNA, 1050 bp, synthetic construct
  misc_feature 1..1050: note = NFAT IL-7 inducible cassette
  source 1..1050: mol_type = other DNA
accggttcaattgccgacccctccccccaacttctcggggactgtgggcgatgtgcgctctgcccactgacgggcaccggagcctcacgatgcatgatatcggcctaactggccggtacctgagctcgctagcggaggaaaaactgtttcatacagaaggcgtggaggaaaaactgtttcatacagaaggcgtggaggaaaaactgtttcatacagaaggcgtgacgtctaggcgtgtacggtgggaggcctatataagcagagctcgtttagtgaaccgtcagatcgcctggagacgccatccacgctgttttgacctccatagaagacaccgggaccgatccagcctctcgacattcgttggatccatatggccgccaccatgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactgactcgagaacttgtttattgcagcttataatggttacaaataaagcaatagcatcacaaatttcacaaataaagcatttttttcactgcattctagttgtggtttgtccaaactcatcaatgtatcttaacgcgt

SEQ ID NO: 2 - DNA, 534 bp, synthetic construct
  misc_feature 1..534: note = codon optimised IL-7
  source 1..534: mol_type = other DNA
atgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactga

SEQ ID NO: 3 - protein, 177 aa, synthetic construct
  REGION 1..177: note = Codon opotimised IL-7
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 4 - DNA, 1854 bp, synthetic construct
  misc_feature 1..1854: note = sequence between Nhel and Sall sites
  source 1..1854: mol_type = other DNA
atggagaccctgctgggcctgctgatcctgtggctgcagctccagtgggtgtccagcaagcaggaggtgacccagatccctgccgccctgagcgtgcccgagggcgagaacctggtgctgaactgcagcttcaccgactccgccatctacaacctgcagtggttccggcaggaccccggcaagggcctgaccagcctgctgctgatccagagcagccagcgggagcagaccagcggacggctgaacgccagcctggacaagagcagcggccggagcaccctgtacatcgccgccagccagcccggcgacagcgccacctacctgtgcgctgtgcggcctctgtacggcggcagctacatccccaccttcggcagaggcaccagcctgatcgtgcacccctacatccagaaccccgaccccgccgtgtaccagctgcgggacagcaagagcagcgacaagtctgtgtgcctgttcaccgacttcgacagccagaccaatgtgagccagagcaaggacagcgacgtgtacatcaccgacaagaccgtgctggacatgcggagcatggacttcaagagcaacagcgccgtggcctggagcaacaagagcgacttcgcctgcgccaacgccttcaacaacagcattatccccgaggacaccttcttccccagccccgagagcagctgcgacgtgaaactggtggagaagagcttcgagaccgacaccaacctgaacttccagaacctgagcgtgatcggcttcagaatcctgctgctgaaggtggccggattcaacctgctgatgaccctgcggctgtggagcagcggctcccgggccaagagaagcggatccggcgccaccaacttcagcctgctgaagcaggccggagacgtggaagaaaaccctggccctaggatgagcatcggcctgctgtgctgcgccgccctgagcctgctgtgggcaggacccgtgaacgccggagtgacccagacccccaagttccaggtgctgaaaaccggccagagcatgaccctgcagtgcgcccaggacatgaaccacgagtacatgagctggtatcggcaggaccccggcatgggcctgcggctgatccactactctgtgggagccggaatcaccgaccagggcgaggtgcccaacggctacaatgtgagccggagcaccaccgaggacttccccctgcggctgctgagcgctgcccccagccagaccagcgtgtacttctgcgccagcagctatgtgggcaacaccggcgagctgttcttcggcgagggctccaggctgaccgtgctggaggacctgaagaacgtgttcccccccgaggtggccgtgttcgagcccagcgaggccgagatcagccacacccagaaggccacactggtgtgtctggccaccggcttctaccccgaccacgtggagctgtcctggtgggtgaacggcaaggaggtgcacagcggcgtgtctaccgacccccagcccctgaaggagcagcccgccctgaacgacagccggtactgcctgtcctccagactgagagtgagcgccaccttctggcagaacccccggaaccacttccggtgccaggtgcagttctacggcctgagcgagaacgacgagtggacccaggaccgggccaagcccgtgacccagattgtgagcgccgaggcctggggcagggccgactgcggcttcaccagcgagagctaccagcagggcgtgctgagcgccaccatcctgtacgagatcctgctgggcaaggccaccctgtacgccgtgctggtgtctgccctggtgctgatggctatggtgaagcggaaggacagccggggctaagtcgac

SEQ ID NO: 5 - protein, 616 aa, synthetic construct
  REGION 1..616: note = TCR1
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKRSGSGATNFSLLKQAGDVEENPGPRMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGV

SEQ ID NO: 6 - protein, 615 aa, synthetic construct
  REGION 1..615: note = TCR1 CDS
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKRSGSGATNFSLLKQAGDVEENPGPRMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 7 - DNA, 4012 bp, synthetic construct
  misc_feature 1..4012: note = sequence spanning hte region between the Mlul and Sall restriction sites.
  source 1..4012: mol_type = other DNA
acgcgttaagatacattgatgagtttggacaaaccacaactagaatgcagtgaaaaaaatgctttatttgtgaaatttgtgatgctattgctttatttgtaaccattataagctgcaataaacaagttctcgagtcagtgctctttggtgcccatcaggatcttgttccagcaggtcttgatttcctgcagcagccgcttcaggaagcacaggtcgttcagtttcttctgctctttcagggacttgttctcttccaggctcttggtaggctgggcttctcccagggcggcaggctttctgcccttcacttggccggtgcaattcagcaggatggtggtgccctcggacactttcagcagatgcaggtcgaagtcgccggtgctgttcatcttcaggaactgccgcagctttctggcggctctgaacaggaacatgccttctttgttggcgtcgcagatgtgccgcttgaagaagttgaactcgttgttcaggcagttgctgccgatttccttcatgctgtccagcagctggtcgatggacaccatcagcacgctctcgtactgcttgccgtccttgccctcgatgtcgcagtcgctgctggccacaggcagcagcaccaggatcagggggggcaggccgaagatgtaccggaaggacacgtggaacatggtggcggccatatggatccaacgaatgtcgagaggctggatcggtcccggtgtcttctatggaggtcaaaacagcgtggatggcgtctccaggcgatctgacggttcactaaacgagctctgcttatataggcctcccaccgtacacgcctagacgtcacgccttctgtatgaaacagtttttcctccacgccttctgtatgaaacagtttttcctccacgccttctgtatgaaacagtttttcctccgctagcgagctcaggtaccggccagttaggccgatatcatgcatcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagccgccaccatggagaccctgctgggcctgctgatcctgtggctgcagctccagtgggtgtccagcaagcaggaggtgacccagatccctgccgccctgagcgtgcccgagggcgagaacctggtgctgaactgcagcttcaccgactccgccatctacaacctgcagtggttccggcaggaccccggcaagggcctgaccagcctgctgctgatccagagcagccagcgggagcagaccagcggacggctgaacgccagcctggacaagagcagcggccggagcaccctgtacatcgccgccagccagcccggcgacagcgccacctacctgtgcgctgtgcggcctctgtacggcggcagctacatccccaccttcggcagaggcaccagcctgatcgtgcacccctacatccagaaccccgaccccgccgtgtaccagctgcgggacagcaagagcagcgacaagtctgtgtgcctgttcaccgacttcgacagccagaccaatgtgagccagagcaaggacagcgacgtgtacatcaccgacaagaccgtgctggacatgcggagcatggacttcaagagcaacagcgccgtggcctggagcaacaagagcgacttcgcctgcgccaacgccttcaacaacagcattatccccgaggacaccttcttccccagccccgagagcagctgcgacgtgaaactggtggagaagagcttcgagaccgacaccaacctgaacttccagaacctgagcgtgatcggcttcagaatcctgctgctgaaggtggccggattcaacctgctgatgaccctgcggctgtggagcagcggctcccgggccaagagaagcggatccggcgccaccaacttcagcctgctgaagcaggccggagacgtggaagaaaaccctggccctaggatgagcatcggcctgctgtgctgcgccgccctgagcctgctgtgggcaggacccgtgaacgccggagtgacccagacccccaagttccaggtgctgaaaaccggccagagcatgaccctgcagtgcgcccaggacatgaaccacgagtacatgagctggtatcggcaggaccccggcatgggcctgcggctgatccactactctgtgggagccggaatcaccgaccagggcgaggtgcccaacggctacaatgtgagccggagcaccaccgaggacttccccctgcggctgctgagcgctgcccccagccagaccagcgtgtacttctgcgccagcagctatgtgggcaacaccggcgagctgttcttcggcgagggctccaggctgaccgtgctggaggacctgaagaacgtgttcccccccgaggtggccgtgttcgagcccagcgaggccgagatcagccacacccagaaggccacactggtgtgtctggccaccggcttctaccccgaccacgtggagctgtcctggtgggtgaacggcaaggaggtgcacagcggcgtgtctaccgacccccagcccctgaaggagcagcccgccctgaacgacagccggtactgcctgtcctccagactgagagtgagcgccaccttctggcagaacccccggaaccacttccggtgccaggtgcagttctacggcctgagcgagaacgacgagtggacccaggaccgggccaagcccgtgacccagattgtgagcgccgaggcctggggcagggccgactgcggcttcaccagcgagagctaccagcagggcgtgctgagcgccaccatcctgtacgagatcctgctgggcaaggccaccctgtacgccgtgctggtgtctgccctggtgctgatggctatggtgaagcggaaggacagccggggctaagtcgac

SEQ ID NO: 8 - DNA, 2484 bp, synthetic construct
  misc_feature 1..2484: note = Consitutive IL-7_T2A_TCR1 covering the region between the Nhel and Sall restriction sites
  source 1..2484: mol_type = other DNA
gctagcgccgccaccatgttccacgtgtccttccggtacatcttcggcctgccccccctgatcctggtgctgctgcctgtggccagcagcgactgcgacatcgagggcaaggacggcaagcagtacgagagcgtgctgatggtgtccatcgaccagctgctggacagcatgaaggaaatcggcagcaactgcctgaacaacgagttcaacttcttcaagcggcacatctgcgacgccaacaaagaaggcatgttcctgttcagagccgccagaaagctgcggcagttcctgaagatgaacagcaccggcgacttcgacctgcatctgctgaaagtgtccgagggcaccaccatcctgctgaattgcaccggccaagtgaagggcagaaagcctgccgccctgggagaagcccagcctaccaagagcctggaagagaacaagtccctgaaagagcagaagaaactgaacgacctgtgcttcctgaagcggctgctgcaggaaatcaagacctgctggaacaagatcctgatgggcaccaaagagcacggaagcagagccaagagaagcggctctggcgagggcagaggcagcctgctgacatgtggcgacgtggaagaaaaccctggccctatggagaccctgctgggcctgctgatcctgtggctgcagctccagtgggtgtccagcaagcaggaggtgacccagatccctgccgccctgagcgtgcccgagggcgagaacctggtgctgaactgcagcttcaccgactccgccatctacaacctgcagtggttccggcaggaccccggcaagggcctgaccagcctgctgctgatccagagcagccagcgggagcagaccagcggacggctgaacgccagcctggacaagagcagcggccggagcaccctgtacatcgccgccagccagcccggcgacagcgccacctacctgtgcgctgtgcggcctctgtacggcggcagctacatccccaccttcggcagaggcaccagcctgatcgtgcacccctacatccagaaccccgaccccgccgtgtaccagctgcgggacagcaagagcagcgacaagtctgtgtgcctgttcaccgacttcgacagccagaccaatgtgagccagagcaaggacagcgacgtgtacatcaccgacaagaccgtgctggacatgcggagcatggacttcaagagcaacagcgccgtggcctggagcaacaagagcgacttcgcctgcgccaacgccttcaacaacagcattatccccgaggacaccttcttccccagccccgagagcagctgcgacgtgaaactggtggagaagagcttcgagaccgacaccaacctgaacttccagaacctgagcgtgatcggcttcagaatcctgctgctgaaggtggccggattcaacctgctgatgaccctgcggctgtggagcagcggctcccgggccaagagaagcggatccggcgccaccaacttcagcctgctgaagcaggccggagacgtggaagaaaaccctggccctaggatgagcatcggcctgctgtgctgcgccgccctgagcctgctgtgggcaggacccgtgaacgccggagtgacccagacccccaagttccaggtgctgaaaaccggccagagcatgaccctgcagtgcgcccaggacatgaaccacgagtacatgagctggtatcggcaggaccccggcatgggcctgcggctgatccactactctgtgggagccggaatcaccgaccagggcgaggtgcccaacggctacaatgtgagccggagcaccaccgaggacttccccctgcggctgctgagcgctgcccccagccagaccagcgtgtacttctgcgccagcagctatgtgggcaacaccggcgagctgttcttcggcgagggctccaggctgaccgtgctggaggacctgaagaacgtgttcccccccgaggtggccgtgttcgagcccagcgaggccgagatcagccacacccagaaggccacactggtgtgtctggccaccggcttctaccccgaccacgtggagctgtcctggtgggtgaacggcaaggaggtgcacagcggcgtgtctaccgacccccagcccctgaaggagcagcccgccctgaacgacagccggtactgcctgtcctccagactgagagtgagcgccaccttctggcagaacccccggaaccacttccggtgccaggtgcagttctacggcctgagcgagaacgacgagtggacccaggaccgggccaagcccgtgacccagattgtgagcgccgaggcctggggcagggccgactgcggcttcaccagcgagagctaccagcagggcgtgctgagcgccaccatcctgtacgagatcctgctgggcaaggccaccctgtacgccgtgctggtgtctgccctggtgctgatggctatggtgaagcggaaggacagccggggctaagtcgac

SEQ ID NO: 9 - protein, 820 aa, synthetic construct
  REGION 1..820: note = Constitutive IL-7_TCR1
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGSRAKRSGSGEGRGSLLTCGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKRSGSGATNFSLLKQAGDVEENPGPRMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 10 - DNA, 4021 bp, synthetic construct
  misc_feature 1..4021: note = Sense strand sequence spanning the region between the Mlul and Sall restriction sites
  source 1..4021: mol_type = other DNA
acgcgttaagatacattgatgagtttggacaaaccacaactagaatgcagtgaaaaaaatgctttatttgtgaaatttgtgatgctattgctttatttgtaaccattataagctgcaataaacaagttctcgagtcagtgctctttggtgcccatcaggatcttgttccagcaggtcttgatttcctgcagcagccgcttcaggaagcacaggtcgttcagtttcttctgctctttcagggacttgttctcttccaggctcttggtaggctgggcttctcccagggcggcaggctttctgcccttcacttggccggtgcaattcagcaggatggtggtgccctcggacactttcagcagatgcaggtcgaagtcgccggtgctgttcatcttcaggaactgccgcagctttctggcggctctgaacaggaacatgccttctttgttggcgtcgcagatgtgccgcttgaagaagttgaactcgttgttcaggcagttgctgccgatttccttcatgctgtccagcagctggtcgatggacaccatcagcacgctctcgtactgcttgccgtccttgccctcgatgtcgcagtcgctgctggccacaggcagcagcaccaggatcagggggggcaggccgaagatgtaccggaaggacacgtggaacatggtggcggccatatggatccaacgaatgtcgagaggctggatcggtcccggtgtcttctatggaggtcaaaacagcgtggatggcgtctccaggcgatctgacggttcactaaacgagctctgcttatataggcctcccaccgtacacgcctagacgtcacgccttctgtatgaaacagtttttcctccacgccttctgtatgaaacagtttttcctccacgccttctgtatgaaacagtttttcctccgctagcgagctcaggtaccggccagttaggccgatatcatgcatcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagccgccaccatgaagaagcacctgaccacctttctcgtgatcctgtggctgtacttctaccggggcaacggcaagaaccaggtggaacagagcccccagagcctgatcatcctggaaggcaagaactgcaccctgcagtgcaactacaccgtgtcccccttcagcaacctgcggtggtacaagcaggacaccggcagaggccctgtgtccctgaccatcctgaccttcagcgagaacaccaagagcaacggccggtacaccgccaccctggacgccgatacaaagcagagcagcctgcacatcaccgccagccagctgagcgatagcgccagctacatctgcgtggtgtccggcggcacagacagctggggcaagctgcagtttggcgccggaacacaggtggtcgtgacccccgacatccagaaccctgaccctgccgtgtaccagctgcgggacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaatagcgccgtggcctggtccaacaagagcgacttcgcctgcgccaacgccttcaacaacagcattatccccgaggacacattcttcccaagccccgagagcagctgcgacgtcaagctggtggaaaagagcttcgagacagacaccaacctgaacttccagaacctgagcgtgatcggcttcagaatcctgctgctgaaggtggccggcttcaacctgctgatgaccctgagactgtggtccagcggcagccgggccaagagatctggatccggcgctaccaactttagcctgctgaagcaggccggggacgtggaagaaaaccctggccctaggatggccagcctgctgttcttctgcggcgccttctacctgctgggcaccggctctatggatgccgacgtgacccagaccccccggaacagaatcaccaagaccggcaagcggatcatgctggaatgctcccagaccaagggccacgaccggatgtactggtacagacaggaccctggcctgggcctgcggctgatctactacagcttcgacgtgaaggacatcaacaagggcgagatcagcgacggctacagcgtgtccagacaggctcaggccaagttcagcctgtccctggaaagcgccatccccaaccagaccgccctgtacttttgtgccacaagcggccagggcgcctacgaggagcagttctttggccctggcacccggctgacagtgctggaagatctgaagaacgtgttccccccagaggtggccgtgttcgagccttctgaggccgaaatcagccacacccagaaagccacactcgtgtgtctggccaccggcttctaccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaaccccagaaaccacttcagatgccaggtgcagttttacggcctgagcgagaacgacgagtggacccaggacagagccaagcccgtgacacagatcgtgtctgccgaagcttgggggcgcgccgattgtggctttaccagcgagagctaccagcagggcgtgctgagcgccaccatcctgtacgagatcctgctgggaaaggccacactgtacgccgtgctggtgtctgccctggtgctgatggccatggtcaagcggaaggacagccggggctaataagtcgac

SEQ ID NO: 11 - protein, 617 aa, synthetic construct
  REGION 1..617: note = TCR2
MKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKRSGSGATNFSLLKQAGDVEENPGPRMASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 12 - DNA, 2512 bp, synthetic construct
  misc_feature 1..2512: note = Sequence covering the region between the Nhel and Sall restriction sites
  source 1..2512: mol_type = other DNA
gctagcgccgccaccatgttccacgtgtccttccggtacatcttcggcctgccccccctgatcctggtgctgctgcctgtggccagcagcgactgcgacatcgagggcaaggacggcaagcagtacgagagcgtgctgatggtgtccatcgaccagctgctggacagcatgaaggaaatcggcagcaactgcctgaacaacgagttcaacttcttcaagcggcacatctgcgacgccaacaaagaaggcatgttcctgttcagagccgccagaaagctgcggcagttcctgaagatgaacagcaccggcgacttcgacctgcatctgctgaaagtgtccgagggcaccaccatcctgctgaattgcaccggccaagtgaagggcagaaagcctgccgccctgggagaagcccagcctaccaagagcctggaagagaacaagtccctgaaagagcagaagaaactgaacgacctgtgcttcctgaagcggctgctgcaggaaatcaagacctgctggaacaagatcctgatgggcaccaaagagcacggaagcagagccaagagaagcggctctggcgcgcccgtgaagcagaccctgaacttcgacctgctgaaactggccggcgacgtggaaagcaaccctggccctatgaagaagcacctgaccacctttctcgtgatcctgtggctgtacttctaccggggcaacggcaagaaccaggtggaacagagcccccagagcctgatcatcctggaaggcaagaactgcactctgcagtgcaactacaccgtgtcccccttcagcaacctgcgctggtacaagcaggataccggcagaggccctgtgtccctgaccatcctgaccttcagcgagaacaccaagagcaacggccggtacaccgccaccctggacgccgatacaaagcagagcagcctgcacatcaccgcctcccagctgagcgatagcgccagctacatctgcgtggtgtccggcggcacagacagctggggcaagctgcagtttggcgccggaacacaggtggtcgtgacccccgacatccagaaccctgaccctgccgtgtaccagctgcgggacagcaagagcagcgacaagagcgtgtgcctgttcaccgacttcgactcccagaccaacgtgtcccagagcaaggacagcgacgtgtacatcaccgacaagaccgtgctggatatgcggagcatggacttcaagagcaatagcgccgtggcctggtctaacaagagcgacttcgcctgcgccaacgccttcaacaacagcattatccccgaggacacattcttcccaagccccgagagcagctgcgacgtgaaactggtggaaaagagcttcgagacagacaccaacctgaatttccagaacctgagcgtgatcggcttccggatcctgctgctgaaggtggccggattcaacctgctgatgaccctgcggctgtggtcctctggctctcgggccaagagaagcggcagcggcgccaccaatttcagcctgctgaagcaggcaggggatgtggaagagaatcccggccctagaatggcctccctgctgtttttctgcggcgccttctacctgctggggaccggcagcatggacgctgacgtgacccagaccccccggaacagaatcaccaagaccggcaagcggatcatgctggaatgcagccagacaaagggccacgaccggatgtactggtacagacaggatccaggactgggcctgaggctgatctactacagcttcgatgtgaaggacatcaacaagggcgagatcagcgacggctacagcgtgtccagacaggcccaggccaagttctccctgagcctggaaagcgccatccccaaccagaccgccctgtacttttgtgccacaagcggccagggcgcctacgaggaacagttctttggccctggcacccggctgacagtgctggaagatctgaagaacgtgttccccccagaggtggcagtgttcgagcctagcgaggccgagatctcccacacccagaaagccacactcgtgtgtctggccaccggattctaccccgaccatgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaaccccagaaatcacttcagatgccaggtgcagttttacggcctgagcgagaacgacgagtggacccaggatagggccaagcccgtgactcagatcgtgtctgccgaagcctggggcagagccgattgcggctttaccagcgagagctaccagcagggcgtgctgagcgccaccatcctgtacgagatcctgctgggcaaggccacactgtacgccgtgctggtgtctgccctggtgctgatggccatggtcaagcggaaggacagccggggctgatgaggtcgac

SEQ ID NO: 13 - protein, 828 aa, synthetic construct
  REGION 1..828: note = Constitutive IL-7_TCR2
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGSRAKRSGSGAPVKQTLNFDLLKLAGDVESNPGPMKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKRSGSGATNFSLLKQAGDVEENPGPRMASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 14 - DNA, 30 bp, synthetic construct
  misc_feature 1..30: note = Human IL2 promoter NFAT TRE
  source 1..30: mol_type = other DNA
ggaggaaaaactgtttcatacagaaggcgt

SEQ ID NO: 15 - DNA, 138 bp, synthetic construct
  misc_feature 1..138: note = Minimal CMV promoter
  source 1..138: mol_type = other DNA
taggcgtgtacggtgggaggcctatataagcagagctcgtttagtgaaccgtcagatcgcctggagacgccatccacgctgttttgacctccatagaagacaccgggaccgatccagcctctcgacattcgttggatc

SEQ ID NO: 16 - DNA, 12 bp, synthetic construct
  misc_feature 1..12: note = Kozak sequence
  source 1..12: mol_type = other DNA
gccgccaccatg

SEQ ID NO: 17 - DNA, 29 bp, synthetic construct
  misc_feature 1..29: note = NHEI_IL_7F
  source 1..29: mol_type = other DNA
taatgctagcgccgccaccatgttccacg

SEQ ID NO: 18 - DNA, 73 bp, synthetic construct
  misc_feature 1..73: note = IL7T2AR
  source 1..73: mol_type = other DNA
cagcaggctgcctctgccctcgccagagccgcttctcttggctctgcttccgtgctctttggtgcccatcagg

SEQ ID NO: 19 - DNA, 20 bp, synthetic construct
  misc_feature 1..20: note = T2AF
  source 1..20: mol_type = other DNA
agggcagaggcagcctgctg

SEQ ID NO: 20 - DNA, 40 bp, synthetic construct
  misc_feature 1..40: note = BETA_SAL_REVIII
  source 1..40: mol_type = other DNA
attattgtcgacttagccccggctgtccttccgcttcacc

SEQ ID NO: 21 - DNA, 34 bp, synthetic construct
  misc_feature 1..34: note = primer
  source 1..34: mol_type = other DNA
taatgctagcgccgccaccatgttccacgtgtcc

SEQ ID NO: 22 - DNA, 32 bp, synthetic construct
  misc_feature 1..32: note = primer
  source 1..32: mol_type = other DNA
cttcacgggcgcgccagagccgcttctcttgg

SEQ ID NO: 23 - DNA, 1054 bp, synthetic construct
  misc_feature 1..1054: note = ADB967 selection
  source 1..1054: mol_type = other DNA
aggcaccggttcaattgccgacccctccccccaacttctcggggactgtgggcgatgtgcgctctgcccactgacgggcaccggagcctcacgatgcatgatatcggcctaactggccggtacctgagctcgctagcggaggaaaaactgtttcatacagaaggcgtggaggaaaaactgtttcatacagaaggcgtggaggaaaaactgtttcatacagaaggcgtgacgtctaggcgtgtacggtgggaggcctatataagcagagctcgtttagtgaaccgtcagatcgcctggagacgccatccacgctgttttgacctccatagaagacaccgggaccgatccagcctctcgacattcgttggatccatatggccgccaccatgttccacgtgtccttccggtacatcttcggcctgccccccctgatcctggtgctgctgcctgtggccagcagcgactgcgacatcgagggcaaggacggcaagcagtacgagagcgtgctgatggtgtccatcgaccagctgctggacagcatgaaggaaatcggcagcaactgcctgaacaacgagttcaacttcttcaagcggcacatctgcgacgccaacaaagaaggcatgttcctgttcagagccgccagaaagctgcggcagttcctgaagatgaacagcaccggcgacttcgacctgcatctgctgaaagtgtccgagggcaccaccatcctgctgaattgcaccggccaagtgaagggcagaaagcctgccgccctgggagaagcccagcctaccaagagcctggaagagaacaagtccctgaaagagcagaagaaactgaacgacctgtgcttcctgaagcggctgctgcaggaaatcaagacctgctggaacaagatcctgatgggcaccaaagagcactgactcgagaacttgtttattgcagcttataatggttacaaataaagcaatagcatcacaaatttcacaaataaagcatttttttcactgcattctagttgtggtttgtccaaactcatcaatgtatcttaacgcgt

SEQ ID NO: 24 - protein, 181 aa, synthetic construct
  REGION 1..181: note = Translation ORF/CDS
MAATMFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

(19) United States
(12) Patent Application Publication
(10) Pub. No.: US 2025/0043011 A1
(43) Pub. Date: Feb. 6, 2025

(54) T Cell Modification and Use Thereof

(71) Applicant: ADAPTIMMUNE LIMITED, Abingdon (GB)

(72) Inventors: Garth Hamilton, Abingdon (GB); Jonathan Silk, Abingdon (GB); Claire Gueguen, Abingdon (GB)

(21) Appl. No.: 18/912,151

(22) Filed: Oct. 10, 2024

Related U.S. Application Data
(63) Continuation of application No. 17/468,225, filed on Sep. 7, 2021, now Pat. No. 12,145,990.
Application No. 17/468,225 is a continuation of application No. 15/998,475, filed on Aug. 15, 2018, now Pat. No. 11,111,301.

(30) Foreign Application Priority Data
Aug. 15, 2017 (GB) 1713078.2

(51) Int. Cl. C07K 16/28; A61K 39/00; A61P 35/00; C07K 14/47; C07K 14/54; C07K 14/725; C07K 16/24; C07K 16/30; C12N 5/0783

(52) U.S. Cl. CPC C07K 16/2833; A61K 39/4611; A61K 39/4631; A61K 39/4632; A61K 39/464481; A61K 39/464486; A61K 39/464488; A61K 39/464489; A61P 35/00; C07K 14/4748; C07K 14/5418; C07K 14/7051; C07K 16/244; C07K 16/30; C12N 5/0636; C07K 2317/622; C07K 2319/03; C07K 2319/33

(57) ABSTRACT

This invention relates to modified T cells that inducibly express a bioactive molecule, such as IL-7, and constitutively expresses an antigen receptor, such as a T cell receptor or chimeric antigen receptor that binds to a tumour antigen. The modified T cells may comprise a nucleic acid construct that comprises a first nucleotide sequence encoding the bioactive molecule, a second nucleotide sequence encoding the antigen receptor; an inducible promoter operably linked to the first nucleotide sequence and a constitutive promoter operably linked to the second nucleotide. Nucleic acid constructs and vectors are provided, as well as T cells comprising such constructs and vectors and therapeutic methods and uses thereof.

### CROSS REFERENCE TO RELATED APPLICATIONS

This application is a continuation of U.S. application Ser. No. 17/468,225, filed Sep. 7, 2021, which is a continuation application of U.S. application Ser. No. 15/998,475, filed Aug. 15, 2018, now U.S. Pat. No. 11,111,301, which claims priority to and the benefit of United Kingdom Application No. GB 1713078.2 filed Aug. 15, 2017, the disclosures of each of which are incorporated herein by reference in their entireties.

### REFERENCE TO AN ELECTRONIC SEQUENCE LISTING

The contents of the electronic sequence listing “341883.xml”, size 52,777 bytes, and Date of Creation Oct. 9, 2024, is herein incorporated by reference in its entirety.

### FIELD

The present invention relates to the modification of T cells to increase their cytotoxic activity and the use of modified T cells in immunotherapy, for example for the treatment of cancer.

### BACKGROUND

T cells (or T lymphocytes) are found widely distributed within tissues and the tumour environment. T cells are distinguished from other lymphocytes by the presence of T cell receptors (TCRs) on the cell surface. The TCR is a multi-subunit transmembrane complex that mediates the antigen-specific activation of T cells. The TCR confers antigen specificity on the T cell, by recognising an antigen peptide ligand that is presented on the target cell by a major histocompatibility complex (MHC) molecule.

Although peptides derived from altered or mutated proteins in tumour target cells can be recognised as foreign by T cells expressing specific TCRs, many antigens on tumour cells are simply upregulated or overexpressed (so called self-antigens) and do not induce a functional T cell response. Therefore, studies have focused on identifying target tumour antigens which are expressed, or highly expressed, in the malignant but not the normal cell type. Examples of such targets include the cancer/testis (CT) antigen NY-ESO-1, which is expressed in a wide array of human cancers but shows restricted expression in normal tissues (Chen Y-T et al. Proc Natl Acad Sci USA. 1997; 94 (5): 1914-1918), and the MAGE-A family of CT antigens which are expressed in a very limited number of healthy tissues (Scanlan M. J. et al. Immunol Rev. 2002; 188:22-32).

Identification of such antigens has promoted the development of targeted T cell-based immunotherapy, which has the potential to provide specific and effective cancer therapy (Ho, W. Y. et al. Cancer Cell 2003; 3:1318-1328; Morris, E. C. et al. Clin. Exp. Immunol. 2003; 131:1-7; Rosenberg, S. A. Nature 2001; 411:380-384; Boon, T. and van der Bruggen P. J. Exp. Med. 1996; 183:725-729).

The intravenous administration of interleukin 7 (IL-7) has been proposed to improve outcomes in T cell-based immunotherapy. IL-7 is known to bolster the persistence of tumour-specific T-cells (Melchionda, F. et al. J. Clin. Invest. 2005; 115:1177-87), and T-cells genetically modified to either secrete IL-7 or overexpress the IL-7 receptor (in conjunction with administered IL-7) display enhanced antitumour efficacy in preclinical models (Vera, J. F. et al. Mol. Ther. 2009; 17:880-8, Markley, J. C. and Sadelain, M. Blood 2010; 115:3508-3519). However, systemic administration of cytokines to patients with cancer has caused significant toxicity (Sportes, C. et al. Clin. Cancer Res. 2010; 16:727-35, Conlon, K. C. t al. J. Clin. Oncol. 2015; 33:74-82, Brudno, J. N. et al. Blood 2016; 127:3321-31) Alternative approaches such as genetic modification of T-cells to secrete or trans-present cytokines (Hutton L. V. et al. Proc. Natl. Acad. Sci. USA 2016; 113: E7788-97) carry a risk of severe adverse events, including neurotoxicity and cytokine release syndrome from systemic accumulation of secreted cytokine (Zhang, L. et al. Clin. Cancer Res. 21; 21:2278-88), whereas T-cells that overexpress cytokine receptors do not eliminate the need for exogenous cytokine (Vera, J. F. et al. Mol. Ther. 2009; 17:880-8). Therefore a method for safely delivering cytokines, such as IL-7, and other bioactive molecules, to T-cells remains elusive.

### SUMMARY

The present inventors have unexpectedly recognised that T cells containing a nucleic acid construct that provides constitutive expression of an antigen receptor and inducible expression of a bioactive molecule, such as Interleukin 7 (IL-7), upon T cell activation, display improved anti-tumour properties without the high toxicity that is observed when the bioactive molecule is expressed constitutively in T cells.

A first aspect of the invention provides a nucleic acid construct comprising:
(i) a first nucleotide sequence encoding a bioactive molecule;
(ii) a second nucleotide sequence encoding an antigen receptor;
(iii) an inducible promoter operably linked to the first nucleotide sequence and
(iv) a constitutive promoter operably linked to the second nucleotide.

Preferably the bioactive molecule is a cytokine, most preferably IL-7.

A second aspect of the invention provides a vector, for example a lentiviral vector, comprising a nucleic construct of the first aspect.

A third aspect of the invention provides a population of T cells comprising a nucleic construct or a vector according to the first or second aspect.

A fourth aspect of the invention provides a population of T cells which constitutively express a heterologous antigen receptor and inducibly express a bioactive molecule, such as IL-7, upon T cell activation.

A fifth aspect of the invention provides a pharmaceutical composition comprising a population of T cells according to the third or fourth aspects and a pharmaceutically acceptable excipient.

A fifth aspect of the invention provides a population of T cells according to the third or fourth aspects for use in a method of treatment of the human or animal body, for example a method of treatment of cancer in an individual. Related aspects provide the use of a population of T cells according to the third or fourth aspects in the manufacture of a medicament for the treatment of cancer in an individual and a method of treating cancer comprising administering to an individual with cancer a population of T cells according to the third or fourth aspects.

A sixth aspect of the invention provides a method of producing a population of modified T cells comprising:
introducing a nucleic acid construct or a vector according to the first or second aspects into a population of T cells obtained from a donor individual to produce a population of modified T cells.

A seventh aspect of the invention provides a method of treating cancer in an individual in need thereof comprising:
introducing a nucleic acid construct or a vector according to the first or second aspects into a population of T cells obtained from a donor individual to produce a population of modified T cells, and
administering the population of modified T cells to a recipient individual.

The donor individual and the recipient individual may be the same (i.e. autologous treatment; the modified T cells are obtained from an individual who is subsequently treated with the modified T cells) or the donor individual and the recipient individual may be different (i.e. allogeneic treatment; the modified T cells are obtained from one individual and subsequently used to treat a different individual).

Other aspects and embodiments of the invention are described below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the sequence and annotation of the NFAT inducible IL-7 expression insert.

The sequence illustrated in a 5′ to 3′ orientation with individual components of the cassette highlighted. These include three copies of the NFAT IL-2 TRE GGAGGAAAAACTGTTTCATACAGAAGGCGT, minimal CMV promoter, IL-7 coding sequence (codon optimised) and the SV40 poly A.

FIG. 2 shows a map of the NFAT-IL-7 plasmid supplied by GeneArt. The NFAT-IL-7 cassette was removed by digestion with AgeI and MluI.

FIG. 3 shows a vector map of the lentiviral vector expressing the TCR1. Highlighted are the unique AgeI and MluI restriction

FIG. 4 shows a vector map of construct ADB967. The NFAT-IL-7 cassette was cloned between the AgeI and MluI restriction sites. Transcription of IL-7 occurs from the antisense strand and in a reverse orientation to that of the TCR1.

FIG. 5 shows a vector map of ADB1099 IL7_T2A_TCR1. IL7_T2A_TCR1 is expressed as a single ORF with IL-7, TCRα and TCRβ chains separated by 2A like skip sequences.

FIG. 6 shows a plasmid map for NFAT inducible IL-7 TCR2

FIG. 7 shows a vector map of constitutive IL-7_TCR2. The IL-7_TCR2 was cloned into the vector AB1224 between the NheI and SalI sites.

FIG. 8 shows a schematic of the design of the three main lentiviral constructs used in this study. Constructs were produced that express IL-7 under the control of 3 repeats of the NFAT promotor derived from the IL-2 promotor sequence in one direction or IL-7 constitutively in the presence SPEAR TCR and constitutive expression of the SPEAR TCR. Note: the sizes of the various components of the constructs are not shown to-scale.

FIG. 9 shows an assessment of the percentage of CD4+ and CD8+ T cells and of T cell transduction efficiency. NTD T cells (white circles), T cells transduced with TCR (black squares), constitutive IL-7_TCR (black triangles) or with NFAT_IL-7_TCR (black circles). Data shows mean+/−SD. (A) The data are shown as the percentage of CD3+ T cells that were either CD4+ or CD8+. (B) The data are shown as the percentage of cells that were CD3+ and either CD4+ or CD8+ and that were TCR Va24+. (C) The data are shown as the mean fluorescence intensity (MFI) in either CD4+ or CD8+Va24+ population.

FIG. 10 shows an assessment of 4-1BB, CD127, CD27, CD28, CD40L, OX-40, PD-1 and TIM-3 expression on CD4+ T cells. NTD T cells (white circles), T cells transduced with TCR (black squares), constitutive IL-7_TCR (black triangles) or with NFAT_IL-7_TCR (black circles). Data shows mean+/−SD. The data are shown as the percentage of cells were either 4-1BB+, CD127+, CD27+, CD28+, CD40L+, OX-40+, PD-1+ or TIM-3+ and either (A) CD4+ (B) CD4+Va24+ or (B) CD4+Va24.

FIG. 11 shows an assessment of 4-1BB, CD127, CD27, CD28, CD40L, OX-40, PD-1 and TIM-3 expression on CD8+ T cells. NTD T cells (white circles), T cells transduced with MAGE-A4c1032 TCR (black squares), constitutive IL-7_MAGE-A4c1032 TCR (black triangles) or with NFAT_IL-7_MAGE-A4c1032TCR (black circles). Data shows mean+/−SD. The data are shown as the percentage of cells were either 41BB+, CD127+, CD27+, CD28+, CD40L+, OX-40+, PD-1+ or TIM-3+ and either (A) CD8+ (B) CD8+Va24+ or (B) CD8+Va24−.

FIG. 12 shows an assessment of the proportions of naïve and memory CD4+ T cells. NTD T cells (white circles), T cells transduced with TCR (black squares), constitutive IL-7_TCR (black triangles) or with NFAT_IL-7_TCR (black circles). Data shows mean+/−SD. The data are shown as the percentage of cells were either CCR7-CD45RA−CD45RO−, CCR7-CD45RA−CD45RO+, CCR7-CD45RA+CD45RO−, CCR7-CD45RA+CD45RO+, CCR7+CD45RA−CD45RO−, CCR7+CD45RA−CD45RO+, CCR7+CD45RA+CD45RO− or CCR7+CD45RA+CD45RO+ and either (A) CD4+ (B) CD4+Va24+ or (B) CD4+Va24−.

FIG. 13 shows an assessment of the proportions of naïve and memory CD8+ T cells. NTD T cells (white circles), T cells transduced with TCR (black squares), constitutive IL-7_TCR (black triangles) or with NFAT_IL-7_TCR (black circles). Data shows mean+/−SD. The data are shown as the percentage of cells were either CCR7-CD45RA−CD45RO−, CCR7-CD45RA−CD45RO+, CCR7-CD45RA+CD45RO−, CCR7-CD45RA+CD45RO+, CCR7+CD45RA−CD45RO−, CCR7+CD45RA−CD45RO+, CCR7+CD45RA+CD45RO− or CCR7+CD45RA+CD45RO+ and either (A) CD8+ (B) CD8+Va24+ or (B) CD8+Va24−.

FIG. 14 shows IL-7 production by T cells in response to antigen positive and negative cells. NTD T cells (white circles), T cells transduced with TCR (black squares), constitutive IL-7_TCR (black triangles) or with NFAT_IL-7_TCR (black circles) were incubated 48 hours alone, or with A375, Mel624 or Colo205 cells. A375 cells pulsed with exogenous peptide or CD3/CD28 beads were included as controls. Data were from 6 donors. Supernatants from T cells were either diluted 1:5 of 1:10. The maximum concentration on the standard curve was 1000 pg/ml and as such any values >2000 pg/ml, 5000 pg/ml or 10.000 pg/ml were assigned a value of 2000 pg/ml, 5000 pg/ml or 10.000 pg/ml respectively. Data shows mean+/−SD of triplicate wells.

FIG. 15 shows total T cell counts from donors FXA-006 and FFA-004 in response to repeated stimulation with irradiated A375 cells (Assay ref. no.: ACF1141). Total live cell counts (Trypan Blue negative cells) from co-cultures with NTD T cells (white circles), T cells transduced with TCR (black squares), constitutive IL-7_TCR (black triangles) or with NFAT_IL-7_TCR (black circles) that were restimulated weekly with irradiated A375 (HLA-A2+/NY-ESO+) cells in the presence or absence of 20 mg/ml IL-7. Restimulation was performed on days 0, 7, 14 and 21 and is indicated by the arrows. T cells were from donors (A) FXA-006 or (B) FFA-004 in the absence of IL-7 or from (C) FXA-006 or (D) FFA-004 in the presence of IL-7. The total cell count was assessed on days 7, 14, 21 and 28.

FIG. 16 shows that the survival of mice injected with T cells co-expressing inducible IL-7 with an engineered TCR compared with T cells expressing TCR alone or TCR together with constitutive IL-7. Survival curves are shown for mice injected i.v. with 1×10⁶ Mel624 tumour cells on day 0, followed by i.v. injection of 2×10⁶ total T cells on day 7 as indicated. The percentage of transduced T cells (TCR VB+/CD3+) were normalised to ˜42% prior to injection using NTD T cells. Mice were culled as poor condition/weight loss required and the graphs indicate the percent survival of each group over the time of the study. (A-E) Percentage survival of mice injected with tumour alone (solid line) or the various T cells as indicated (dashed and dotted lines).

### DETAILED DESCRIPTION

This invention relates to modified T cells that inducibly express a bioactive molecule, such as IL-7, and constitutively expresses an antigen receptor. The modified T cells may comprise a nucleic acid construct that comprises;
(i) a first nucleotide sequence encoding a bioactive molecule
(ii) a second nucleotide sequence encoding an antigen receptor;
(iii) an inducible promoter operably linked to the first nucleotide sequence and
(iv) a constitutive promoter operably linked to the second nucleotide.

T cells (also called T lymphocytes) are white blood cells that play a central role in cell-mediated immunity. T cells can be distinguished from other lymphocytes by the presence of a T cell receptor (TCR) on the cell surface. There are several types of T cells, each type having a distinct function.

T helper cells (TH cells) are known as CD⁴⁺ T cells because they express the CD⁴⁺ surface glycoprotein. CD⁴⁺ T cells play an important role in the adaptive immune system and help the activity of other immune cells by releasing T cell cytokines and helping to suppress or regulate immune responses. They are essential for the activation and growth of cytotoxic T cells.

Cytotoxic T cells (Tc cells, CTLs, killer T cells) are known as CD⁸⁺ T cells because they express the CD⁸⁺ surface glycoprotein. CD⁸⁺ T cells act to destroy virus-infected cells and tumour cells. Most CD⁸⁺ T cells express TCRs that can recognise a specific antigen displayed on the surface of infected or damaged cells by a class I MHC molecule. Specific binding of the TCR and CD⁸⁺ glycoprotein to the antigen and MHC molecule leads to T cell-mediated destruction of the infected or damaged cells.

T cells for use as described herein may be CD⁴⁺ T cells; CD⁸⁺ T cells; or CD⁴⁺ T cells and CD⁸⁺ T cells. For example, the T cells may be a mixed population of CD⁴⁺ T cells and CD⁸⁺ T cells.

Suitable T cells for use as described herein may be obtained from a donor individual. In some embodiments, the donor individual may be the same person as the recipient individual to whom the T cells will be administered following modification and expansion as described herein (autologous treatment). In other embodiments, the donor individual may be a different person to the recipient individual to whom the T cells will be administered following modification and expansion as described herein (allogeneic treatment). For example, the donor individual may be a healthy individual who is human leukocyte antigen (HLA) matched (either before or after donation) with a recipient individual suffering from cancer.

A method described herein may comprise the step of obtaining T cells from a donor individual and/or isolating T cells from a sample obtained from a donor individual with cancer.

A population of T cells may be isolated from a blood sample. Suitable methods for the isolation of T cells are well known in the art and include, for example fluorescent activated cell sorting (FACS: see for example, Rheinherz et al (1979) PNAS 76 4061), cell panning (see for example, Lum et al (1982) Cell Immunol 72 122) and isolation using antibody coated magnetic beads (see, for example, Gaudernack et al 1986 J Immunol Methods 90 179). CD4 and CD8 T cells may be isolated from the population of peripheral blood mononuclear cells (PBMCs) obtained from a blood sample. PBMCs may be extracted from a blood sample using standard techniques. For example, ficoll may be used in combination with gradient centrifugation (Böyum A. Scand J Clin Lab Invest. 1968; 21 (Suppl.97): 77-89), to separate whole blood into a top layer of plasma, followed by a layer of PBMCs and a bottom fraction of polymorphonuclear cells and erythrocytes. In some embodiments, the PBMCs may be depleted of CD14 cells (monocytes).

Following isolation, the T cells may be activated. Suitable methods for activating T cells are well known in the art. For example, the isolated T cells may be exposed to a T cell receptor (TCR) agonist. Suitable TCR agonists include ligands, such as a peptide displayed on a class I or II MHC molecule on the surface of an antigen presenting cell, such as a dendritic cell, and soluble factors, such as anti-TCR antibodies.

An anti-TCR antibody may specifically bind to a component of the TCR, such as εCD3, αCD3 or αCD28. Anti-TCR antibodies suitable for TCR stimulation are well-known in the art (e.g. OKT3) and available from commercial suppliers (e.g. eBioscience CO USA). In some embodiments, T cells may be activated by exposure to anti-αCD3 antibodies and IL2. More preferably, T cells are activated by exposure to anti-αCD3 antibodies and anti-αCD28 antibodies. The activation may occur in the presence or absence of CD14 monocytes. Preferably, the T cells may be activated with anti-CD3 and anti-CD28 antibody coated beads. For example, PBMCs or T cell subsets including CD4 and/or CD8 cells may be activated, without feeder cells (antigen presenting cells) or antigen, using antibody coated beads, for example magnetic beads coated with anti-CD3 and anti-CD28 antibodies, such as Dynabeads® Human T-Activator CD3/CD28 (ThermoFisher Scientific).

Following isolation and activation, the T cells may be modified to incorporate the nucleic acid construct.

The bioactive molecule and antigen receptor expressed in the modified T cell are recombinant proteins that are encoded by heterologous nucleic acid i.e. the bioactive molecule and the antigen receptor are expressed from encoding nucleic acid that has been incorporated into the T cell by recombinant techniques.

Modification of a T cell to express the bioactive molecule and the antigen receptor may comprise introducing the nucleic acid construct into the T cell. Suitable methods for the introduction and expression of heterologous nucleic acids into T cells are well-known in the art and described in more detail below.

The bioactive molecule may be a growth factor or a cytokine, preferably Interleukin 7 (IL-7). Interleukin 7 (IL-7) may be human IL-7 and may have the amino acid sequence of SEQ ID NO: 3.

Expression of IL-7 from the inducible promoter is induced by T-cell activation.

The inducible promoter may comprise a nuclear factor of activated T cells (NFAT)/AP1 transcriptional response element (TRE). Upon recognition of the cognate peptide MHC1 complex, NFAT undergoes Ca2+ dependent translocation to the nucleus where it promotes transcription of genes which harbour an NFAT TRE. Suitable NFAT TREs are well-known in the art and include the human IL2 promoter NFAT TRE (Macian et al (2001) Oncogene. 2001 Apr. 30; 20 (19): 2476-89) which has the sequence of SEQ ID NO: 14 or a variant thereof.

The inducible promoter may comprise one, two, three or more repeats of the NFAT TRE.

The inducible promoter may further comprise additional promoter elements, for example a minimal viral promoter such as CMV. Suitable promoter elements are well known in the art and include the minimal CMV promoter of SEQ ID NO: 15 or a variant thereof.

A suitable inducible promoter sequence operably linked to a nucleotide sequence encoding IL-7 may comprise the nucleotide sequence of SEQ ID NO: 1 or a variant thereof

Expression from the constitutive promoter does not vary in response to transcription factors and the second nucleic acid sequence is expressed continuously in the T cell. Suitable constitutive promoters are well known in the art and include mammalian promoters, such as Human elongation factor-1 alpha (EF1α).

A suitable antigen receptor may bind specifically to target cells, preferably cancer cells.

The antigen receptor may be a T cell receptor (TCR). TCRs are disulphide-linked membrane anchored heterodimeric proteins, typically comprising highly variable alpha (a) and beta (β) chains expressed as a complex with invariant CD3 chain molecules. T cells expressing these type of TCRs are referred to as α:β (or αβ) T cells. A minority of T cells express an alternative TCR comprising variable gamma (γ) and delta (δ) chains and are referred to as γδ T cells.

Suitable TCRs bind specifically to a major histocompatibility complex (MHC) on the surface of cancer cells that displays a peptide fragment of a tumour antigen. An MHC is a set of cell-surface proteins which allow the acquired immune system to recognise ‘foreign’ molecules. Proteins are intracellularly degraded and presented on the surface of cells by the MHC. MHCs displaying ‘foreign’ peptides, such a viral or cancer associated peptides, are recognised by T cells with the appropriate TCRs, prompting cell destruction pathways. MHCs on the surface of cancer cells may display peptide fragments of tumour antigen i.e. an antigen which is present on a cancer cell but not the corresponding non-cancerous cell. T cells which recognise these peptide fragments may exert a cytotoxic effect on the cancer cell.

Suitable TCRs are well known in the art and include the TCRs of SEQ ID NOs: 6 and 11 and variants thereof.

In some embodiments, the coding sequences for the individual chains of the TCR (e.g. TCRα and TCRβ chains) may be separated by a cleavage recognition sequence. This allows the chains of the TCR to be expressed as a single fusion which undergoes intracellular cleavage to generate the two separate proteins. Suitable cleavage recognition sequences are well known in the art and include 2A-furin sequence.

Preferably, the TCR is not naturally expressed by the T cells (i.e. the TCR is exogenous or heterologous). Heterologous TCRs may include αβTCR heterodimers. Suitable heterologous TCRs may bind specifically to cancer cells that express a tumour antigen. For example, the T cells may be modified to express a heterologous TCR that binds specifically to MHCs displaying peptide fragments of a tumour antigen expressed by the cancer cells in a specific cancer patient. Tumour antigens expressed by cancer cells in the cancer patient may identified using standard techniques.

A heterologous TCR may be a synthetic or artificial TCR i.e. a TCR that does not exist in nature. For example, a heterologous TCR may be engineered to increase its affinity or avidity for a tumour antigen (i.e. an affinity enhanced TCR). The affinity enhanced TCR may comprise one or more mutations relative to a naturally occurring TCR, for example, one or more mutations in the hypervariable complementarity determining regions (CDRs) of the variable regions of the TCR α and β chains. These mutations increase the affinity of the TCR for MHCs that display a peptide fragment of a tumour antigen expressed by cancer cells. Suitable methods of generated affinity enhanced TCRs include screening libraries of TCR mutants using phage or yeast display and are well known in the art (see for example Robbins et al J Immunol (2008) 180 (9): 6116; San Miguel et al (2015) Cancer Cell 28 (3) 281-283; Schmitt et al (2013) Blood 122 348-256; Jiang et al (2015) Cancer Discovery 5 901).

Preferred affinity enhanced TCRs may bind to cancer cells expressing one or more of the tumour antigens NY-ESO1, PRAME, alpha-fetoprotein (AFP), MAGE A4, MAGE A1, MAGE A10 and MAGE B2.

Alternatively, the antigen receptor may be a chimeric antigen receptor (CAR). CARs are artificial receptors that are engineered to contain an immunoglobulin antigen binding domain, such as a single-chain variable fragment (scFv). A CAR may, for example, comprise an scFv fused to a TCR CD3 transmembrane region and endodomain. An scFv is a fusion protein of the variable regions of the heavy (V_{H}) and light (V_{L}) chains of immunoglobulins, which may be connected with a short linker peptide of approximately 10 to 25 amino acids (Huston J. S. et al. Proc Natl Acad Sci USA 1988; 85 (16): 5879-5883). The linker may be glycine-rich for flexibility, and serine or threonine rich for solubility, and may connect the N-terminus of the V_{H} to the C-terminus of the V_{L}, or vice versa. The scFv may be preceded by a signal peptide to direct the protein to the endoplasmic reticulum, and subsequently the T cell surface. In the CAR, the scFv may be fused to a TCR transmembrane and endodomain. A flexible spacer may be included between the scFv and the TCR transmembrane domain to allow for variable orientation and antigen binding. The endodomain is the functional signal-transmitting domain of the receptor. An endodomain of a CAR may comprise, for example, intracellular signalling domains from the CD3 ζ-chain, or from receptors such as CD28, 41BB, or ICOS. A CAR may comprise multiple signalling domains, for example, but not limited to, CD3z-CD28-41BB or CD3z-CD28-OX40.

The CAR may bind specifically to a tumour-specific antigen expressed by cancer cells. For example, the T cells may be modified to express a CAR that binds specifically to a tumour antigen that is expressed by the cancer cells in a specific cancer patient. Tumour antigens expressed by cancer cells in the cancer patient may identified using standard techniques.

Expression of a heterologous antigen receptor, such as a heterologous TCR or CAR, may alter the immunogenic specificity of the T cells so that they recognise or display improved recognition for one or more tumour antigens that are present on the surface of the cancer cells of an individual with cancer.

In some embodiments, the T cells may display reduced binding or no binding to cancer cells in the absence of the heterologous antigen receptor. For example, expression of the heterologous antigen receptor may increase the affinity and/or specificity of the cancer cell binding of modified T cells relative to unmodified T cells.

The term “heterologous” refers to a polypeptide or nucleic acid that is foreign to a particular biological system, such as a host cell, and is not naturally present in that system. A heterologous polypeptide or nucleic acid may be introduced to a biological system by artificial means, for example using recombinant techniques. For example, heterologous nucleic acid encoding a polypeptide may be inserted into a suitable expression construct which is in turn used to transform a host cell to produce the polypeptide. A heterologous polypeptide or nucleic acid may be synthetic or artificial or may exist in a different biological system, such as a different species or cell type. An endogenous polypeptide or nucleic acid is native to a particular biological system, such as a host cell, and is naturally present in that system. A recombinant polypeptide is expressed from heterologous nucleic acid that has been introduced into a cell by artificial means, for example using recombinant techniques. A recombinant polypeptide may be identical to a polypeptide that is naturally present in the cell or may be different from the polypeptides that are naturally present in that cell.

A variant of a reference amino acid or nucleotide sequence set out herein may comprise a sequence having at least 50%, at least 60%, at least 70%, at least 80%, at least 90%, at least 95%, or at least 98% sequence identity to the reference sequence. Particular amino acid sequence variants may differ from a repeat domain shown above by insertion, addition, substitution or deletion of 1 amino acid, 2, 3, 4, 5, 6, 7, 8, 9, or 10 or more than 10 amino acids. Particular nucleotide sequence variants may differ from a reference sequence set out herein by insertion, addition, substitution or deletion of 1 amino acid, 2, 3, 4, 5, 6, 7, 8, 9, or 10 or more than 10 amino acids.

Sequence similarity and identity are commonly defined with reference to the algorithm GAP (Wisconsin Package, Accelerys, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215:405-410), FASTA (which uses the method of Pearson and Lipman (1988) PNAS USA 85:2444-2448), or the Smith-Waterman algorithm (Smith and Waterman (1981) J. Mol Biol. 147:195-197), or the TBLASTN program, of Altschul et al. (1990) supra, generally employing default parameters. In particular, the psi-Blast algorithm (Nucl. Acids Res. (1997) 25 3389-3402) may be used.

Sequence comparison may be made over the full-length of the relevant sequence described herein.

The heterologous antigen receptor encoded by the second nucleotide sequence may specifically bind to the cancer cells of a cancer patient. The cancer patient may be subsequently treated with the modified T cells. Suitable cancer patients for treatment with the modified T cells may be identified by a method comprising:
obtaining sample of cancer cells from an individual with cancer and;
identifying the cancer cells as binding to the antigen receptor encoded by the second nucleotide sequence and expressed by the modified T cells.

Cancer cells may be identified as binding to the antigen receptor encoded by the second nucleotide sequence by identifying one or more tumour antigens expressed by the cancer cells. Methods of identifying antigens on the surface of cancer cells obtained from an individual with cancer are well-known in the art.

In some embodiments, a heterologous antigen receptor suitable for the treatment of a specific cancer patient may be identified by;
obtaining sample of cancer cells from an individual with cancer and;
identifying an antigen receptor that specifically binds to the cancer cells.

An antigen receptor that specifically binds to the cancer cells may be identified for example by identifying one or more tumour antigens expressed by the cancer cells. Methods of identifying antigens on the surface of cancer cells obtained from an individual with cancer are well-known in the art. An antigen receptor which binds to the one or more tumour antigens or which binds to MHC-displayed peptide fragments of the one or more antigens may then be identified, for example from antigen receptors of known specificities or by screening a panel or library of antigen receptors with diverse specificities. Antigen receptors that specifically bind to cancer cells having one or more defined tumour antigens may be produced using routine techniques.

Nucleic acid encoding the identified antigen receptor may be used as the second nucleotide sequence in a nucleic acid construct as described herein.

The cancer cells of an individual suitable for treatment as described herein may express the antigen and may be of correct HLA type to bind the antigen receptor.

Cancer cells may be distinguished from normal somatic cells in an individual by the expression of one or more antigens (i.e. tumour antigens). Normal somatic cells in an individual may not express the one or more antigens or may express them in a different manner, for example at lower levels, in different tissue and/or at a different developmental stage. Tumour antigens may elicit immune responses in the individual. In particular, a tumour antigen may elicit a T cell-mediated immune response against cancer cells in the individual that express the tumour antigen. One or more tumour antigens expressed by cancer cells in a patient may be selected as a target antigen for heterologous receptors on modified T cells.

Tumour antigens expressed by cancer cells may include, for example, cancer-testis (CT) antigens encoded by cancer-germ line genes, such as MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-I, GAGE-2, GAGE-3, GAGE-4, GAGE-5, GAGE-6, GAGE-7, GAGE-8, BAGE-I, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-I, LAGE-I, SSX-I, SSX-2 (HOM-MEL-40), SSX-3, SSX-4, SSX-5, SCP-I and XAGE and immunogenic fragments thereof (Simpson et al. Nature Rev (2005) 5, 615-625, Gure et al., Clin Cancer Res (2005) 11, 8055-8062; Velazquez et al., Cancer Immun (2007) 7, 1 1; Andrade et al., Cancer Immun (2008) 8, 2; Tinguely et al., Cancer Science (2008); Napoletano et al., Am J of Obstet Gyn (2008) 198, 99 e91-97).

Other tumour antigens include, for example, overexpressed, upregulated or mutated proteins and differentiation antigens particularly melanocyte differentiation antigens such as p53, ras, CEA, MUC1, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-All, hsp70-2, KIAAO205, Mart2, Mum-2, and 3, neo-PAP, myosin class I, OS-9, pml-RAR.alpha. fusion protein, PTPRK, K-ras, N-ras, Triosephosphate isomerase, GnTV, Herv-K-mel, NA-88, SP17, and TRP2-Int2, (MART-I), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM17.1, NuMa, K-ras, alpha.-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding protein\cyclophilin C-associated protein), TAAL6, TAG72, TLP, TPS and tyrosinase related proteins such as TRP-1, TRP-2.

Other tumour antigens include out-of-frame peptide-MHC complexes generated by the non-AUG translation initiation mechanisms employed by “stressed” cancer cells (Malarkannan et al. Immunity 1999 June; 10 (6): 681-90).

Other tumour antigens are well-known in the art (see for example WO00/20581; Cancer Vaccines and Immunotherapy (2000) Eds Stern, Beverley and Carroll, Cambridge University Press, Cambridge) The sequences of these tumour antigens are readily available from public databases but are also found in WO 1992/020356 A1, WO 1994/005304 A1, WO 1994/023031 A1, WO 1995/020974 A1, WO 1995/023874 A1 and WO 1996/026214 A1.

Preferred tumour antigens include NY-ESO1, PRAME, alpha-fetoprotein (AFP), MAGE A4, MAGE A1, MAGE A10 and MAGE B2, most preferably NY-ESO-1 and MAGE-A10.

NY-ESO-1 is a human tumour antigen of the cancer/testis (CT) family and is frequently expressed in a wide variety of cancers, including melanoma, prostate, transitional cell bladder, breast, lung, thyroid, gastric, head and neck, and cervical carcinoma (van Rhee F. et al. Blood 2005; 105 (10): 3939-3944). In addition, expression of NY-ESO-1 is usually limited to germ cells and is not expressed in somatic cells (Scanlan M. J. et al. Cancer Immun. 2004; 4 (1)). Suitable affinity enhanced TCRs that bind to cancer cells expressing NY-ESO-1 include NY-ESO-1 c²⁵⁹.

NY-ESO-1 c²⁵⁹ is an affinity enhanced TCR is mutated at positions 95 and 96 of the alpha chain 95:96LY relative to the wildtype TCR. NY-ESO-1 c²⁵⁹ binds to a peptide corresponding to amino acid residues 157-165 of the human cancer testis Ag NY-ESO-1 (SLLMWITQC) in the context of the HLA-A2+ class 1 allele with increased affinity relative to the unmodified wild type TCR (Robbins et al J Immunol (2008) 180 (9): 6116).

MAGE-A10 is a highly immunogenic member of the MAGE-A family of CT antigens, and is expressed in germ cells but not in healthy tissue. MAGE-A10 is expressed in high percentages of cancer cells from a number of tumours (Schultz-Thater E. et al. Int J Cancer. 2011; 129 (5): 1137-1148).

The introduction of the nucleic acid construct into T cells and their subsequent expansion may be performed in vitro and/or ex vivo.

The first nucleotide sequence encoding the bioactive molecule and the second nucleotide sequence encoding the antigen receptor are introduced into the T cells in the same expression vector. This increases the proportion of T cells which express both genes after transduction.

The first nucleotide sequence may be configured for expression in a first direction and the second nucleotide sequence may be configured for expression in a second direction in the nucleic acid construct. For example, the first nucleotide sequence encoding IL-7 may be in the forward orientation in the nucleic acid construct and the second nucleotide sequence encoding the antigen receptor may be in the reverse orientation or the first nucleotide sequence encoding IL-7 may be in the reverse orientation and the second nucleotide sequence encoding the antigen receptor may be in the forward orientation. The first nucleotide sequence may be transcribed from the sense strand of the nucleic acid construct and the second nucleotide sequence may be transcribed from the antisense strand of the nucleic acid construct or the first nucleotide sequence may be transcribed from the antisense strand of the nucleic acid construct and the second nucleotide sequence may be transcribed from the sense strand of the nucleic acid construct.

The nucleic acid construct may include one or more unique restriction sites to facilitate further manipulation.

In some embodiments, the nucleic acid construct may be introduced directly until T cells using gene editing techniques.

In other embodiments, the nucleic acid construct may be incorporated into an expression vector. Suitable vectors are well known in the art and are described in more detail herein.

Examples of suitable vectors include AB1581 and ADB967 as described below. Suitable vectors can be chosen or constructed, containing appropriate regulatory sequences, including promoter sequences, terminator fragments, polyadenylation sequences, enhancer sequences, marker genes and other sequences as appropriate. Preferably, the vector contains appropriate regulatory sequences to drive the expression of the nucleic acid in mammalian cells. A vector may also comprise sequences, such as origins of replication, promoter regions and selectable markers, which allow for its selection, expression and replication in bacterial hosts such as E. coli.

Preferably, the nucleic acid construct is contained in a viral vector, most preferably a gamma retroviral vector or a lentiviral vector, such as a VSVg-pseudotyped lentiviral vector. The T cells may be transduced by contact with a viral particle comprising the nucleic acid. Viral particles for transduction may be produced according to known methods. For example, HEK293T cells may be transfected with plasmids encoding viral packaging and envelope elements as well as a lentiviral vector comprising the coding nucleic acid. A VSVg-pseudotyped viral vector may be produced in combination with the viral envelope glycoprotein G of the Vesicular stomatitis virus (VSVg) to produce a pseudotyped virus particle

A viral vector, such as a lentivirus, may be contained in a viral particle comprising the nucleic acid vector encapsulated by one or more viral proteins. A viral particle may be produced by a method comprising transducing mammalian cells with a viral vector as described herein and one or more viral packaging and envelope vectors and culturing the transduced cells in a culture medium, such that the cells produce lentiviral particles that are released into the medium.

Following release of viral particles, the culture medium comprising the viral particles may be collected and, optionally the viral particles may be concentrated.

Following production and optional concentration, the viral particles may be stored, for example by freezing at −80° C. ready for use in transducing T cells.

The nucleic acid construct or vector may be introduced into the T cells by any convenient method. When introducing or incorporating a heterologous nucleic acid into a T cell, certain considerations must be taken into account, well-known to those skilled in the art. The nucleic acid to be inserted should be assembled within a construct or vector which contains effective regulatory elements which will drive transcription in the T cell. Suitable techniques for transporting the constructor vector into the T cell are well known in the art and include calcium phosphate transfection, DEAE-Dextran, electroporation, liposome-mediated transfection and transduction using retrovirus or other virus, e.g. vaccinia or lentivirus. For example, solid-phase transduction may be performed without selection by culture on retronectin-coated, retroviral vector-preloaded tissue culture plates.

Many known techniques and protocols for manipulation and transformation of nucleic acid, for example in preparation of nucleic acid constructs, introduction of DNA into cells and gene expression are described in detail in Protocols in Molecular Biology, Second Edition, Ausubel et al. eds. John Wiley & Sons, 1992.

Following the introduction of nucleic acid into the T cells, the initial population of modified T cells may be cultured in vitro such that the modified T cells proliferate and expand the population.

The modified T cell population may for example be expanded using magnetic beads coated with anti-CD3 and anti-CD28. The modified T cells may be cultured using any convenient technique to produce the expanded population. Suitable culture systems include stirred tank fermenters, airlift fermenters, roller bottles, culture bags or dishes, and other bioreactors, in particular hollow fibre bioreactors. The use of such systems is well-known in the art.

Numerous culture media suitable for use in the proliferation of T cells ex vivo are available, in particular complete media, such as AIM-V, Iscoves medium and RPMI-1640 (Invitrogen-GIBCO). The medium may be supplemented with other factors such as serum, serum proteins and selective agents. For example, in some embodiments, RPMI-1640 medium containing 2 mM glutamine, 10% FBS, 25 mM HEPES, pH 7.2, 1% penicillin-streptomycin, and 55 μM β-mercaptoethanol and optionally supplemented with 20 ng/ml recombinant IL-2 may be employed. The culture medium may be supplemented with the agonistic or antagonist factors described above at standard concentrations which may readily be determined by the skilled person by routine experimentation.

Conveniently, cells are cultured at 37° C. in a humidified atmosphere containing 5% CO₂ in a suitable culture medium.

Methods and techniques for the culture of T cells and other mammalian cells are well-known in the art (see, for example, Basic Cell Culture Protocols, C. Helgason, Humana Press Inc. U.S. (15 Oct. 2004) ISBN: 1588295451; Human Cell Culture Protocols (Methods in Molecular Medicine S.) Humana Press Inc., U.S. (9 Dec. 2004) ISBN: 1588292223; Culture of Animal Cells: A Manual of Basic Technique, R. Freshney, John Wiley & Sons Inc (2 Aug. 2005) ISBN: 0471453293, Ho W Y et al J Immunol Methods. (2006) 310:40-52)

In some embodiments, it may be convenient to isolate and/or purify the modified T cells from the population. Any convenient technique may be used, including FACS and antibody coated magnetic particles.

Optionally, the population of modified T cells produced as described herein may be stored, for example by lyophilisation and/or cryopreservation, before use.

A population of modified T cells may be admixed with other reagents, such as buffers, carriers, diluents, preservatives and/or pharmaceutically acceptable excipients. Suitable reagents are described in more detail below. A method described herein may comprise admixing the population of modified T cells with a pharmaceutically acceptable excipient.

Pharmaceutical compositions suitable for administration (e.g. by infusion), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents. Examples of suitable isotonic vehicles for use in such formulations include Sodium Chloride Injection, Ringer's Solution, or Lactated Ringer's Injection. Suitable vehicles can be found in standard pharmaceutical texts, for example, Remington's Pharmaceutical Sciences, 18th edition, Mack Publishing Company, Easton, Pa., 1990.

In some preferred embodiments, the modified T cells may be formulated into a pharmaceutical composition suitable for intravenous infusion into an individual.

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Other aspects of the invention provide a population of modified T cells expressing a nucleic construct or a vector as described herein and a population of T cells that constitutively express a heterologous antigen receptor and express IL-7 inducibly upon T cell activation.

The T cells may bind specifically to cancer cells. A suitable population may be produced by a method described above.

The population of modified T cells may be for use as a medicament. For example, a population of modified T cells as described herein may be used in cancer immunotherapy therapy, for example adoptive T cell therapy.

Other aspects of the invention provide the use of a population of modified T cells as described herein for the manufacture of a medicament for the treatment of cancer, a population of modified T cells as described herein for the treatment of cancer, and a method of treatment of cancer may comprise administering a population of modified T cells as described herein to an individual in need thereof.

The population of modified T cells may be autologous i.e. the modified T cells were originally obtained from the same individual to whom they are subsequently administered (i.e. the donor and recipient individual are the same). A suitable population of modified T cells for administration to the individual may be produced by a method comprising providing an initial population of T cells obtained from the individual, modifying the T cells to inducibly express IL-7 and constitutively express an antigen receptor which binds specifically to cancer cells in the individual as described herein, and culturing the modified T cells.

The population of modified T cells may be allogeneic i.e. the modified T cells were originally obtained from a different individual to the individual to whom they are subsequently administered (i.e. the donor and recipient individual are different). The donor and recipient individuals may be HLA matched to avoid GVHD and other undesirable immune effects. A suitable population of modified T cells for administration to a recipient individual may be produced by a method comprising providing an initial population of T cells obtained from a donor individual, modifying the T cells to inducibly express IL-7 and constitutively express an antigen receptor which binds specifically to cancer cells in the recipient individual, as described herein, and culturing the modified T cells.

Following administration of the modified T cells, the recipient individual may exhibit a T cell mediated immune response against cancer cells in the recipient individual. This may have a beneficial effect on the cancer condition in the individual.

Cancer conditions may be characterised by the abnormal proliferation of malignant cancer cells and may include leukaemias, such as AML, CML, ALL and CLL, lymphomas, such as Hodgkin lymphoma, non-Hodgkin lymphoma and multiple myeloma, and solid cancers such as sarcomas, skin cancer, melanoma, bladder cancer, brain cancer, breast cancer, uterus cancer, ovary cancer, prostate cancer, lung cancer, colorectal cancer, cervical cancer, liver cancer, head and neck cancer, oesophageal cancer, pancreas cancer, renal cancer, adrenal cancer, stomach cancer, testicular cancer, cancer of the gall bladder and biliary tracts, thyroid cancer, thymus cancer, cancer of bone, and cerebral cancer, as well as cancer of unknown primary (CUP).

Cancer cells within an individual may be immunologically distinct from normal somatic cells in the individual (i.e. the cancerous tumour may be immunogenic). For example, the cancer cells may be capable of eliciting a systemic immune response in the individual against one or more antigens expressed by the cancer cells. The tumour antigens that elicit the immune response may be specific to cancer cells or may be shared by one or more normal cells in the individual.

An individual suitable for treatment as described above may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orang-utan, gibbon), or a human.

In preferred embodiments, the individual is a human. In other preferred embodiments, non-human mammals, especially mammals that are conventionally used as models for demonstrating therapeutic efficacy in humans (e.g. murine, primate, porcine, canine, or rabbit animals) may be employed.

In some embodiments, the individual may have minimal residual disease (MRD) after an initial cancer treatment.

An individual with cancer may display at least one identifiable sign, symptom, or laboratory finding that is sufficient to make a diagnosis of cancer in accordance with clinical standards known in the art. Examples of such clinical standards can be found in textbooks of medicine such as Harrison's Principles of Internal Medicine, 15th Ed., Fauci A S et al., eds., McGraw-Hill, New York, 2001. In some instances, a diagnosis of a cancer in an individual may include identification of a particular cell type (e.g. a cancer cell) in a sample of a body fluid or tissue obtained from the individual.

Treatment may be any treatment and therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Treatment may also be prophylactic (i.e. prophylaxis). For example, an individual susceptible to or at risk of the occurrence or re-occurrence of cancer may be treated as described herein. Such treatment may prevent or delay the occurrence or re-occurrence of cancer in the individual.

In particular, treatment may include inhibiting cancer growth, including complete cancer remission, and/or inhibiting cancer metastasis. Cancer growth generally refers to any one of a number of indices that indicate change within the cancer to a more developed form. Thus, indices for measuring an inhibition of cancer growth include a decrease in cancer cell survival, a decrease in tumour volume or morphology (for example, as determined using computed tomographic (CT), sonography, or other imaging method), a delayed tumour growth, a destruction of tumour vasculature, improved performance in delayed hypersensitivity skin test, an increase in the activity of T cells, and a decrease in levels of tumour-specific antigens. Administration of T cells modified as described herein may improve the capacity of the individual to resist cancer growth, in particular growth of a cancer already present the subject and/or decrease the propensity for cancer growth in the individual.

The modified T cells or the pharmaceutical composition comprising the modified T cells may be administered to a subject by any convenient route of administration, whether systemically/peripherally or at the site of desired action, including but not limited to; parenteral, for example, by infusion. Infusion involves the administration of the T cells in a suitable composition through a needle or catheter. Typically, T cells are infused intravenously or subcutaneously, although the T cells may be infused via other non-oral routes, such as intramuscular injections and epidural routes. Suitable infusion techniques are known in the art and commonly used in therapy (see, e.g., Rosenberg et al., New Eng. J. of Med., 319:1676, 1988).

Typically, the number of cells administered is from about 10⁵ to about 10¹⁰ per Kg body weight, typically 2×10⁸ to 2×10¹⁰ cells per individual, typically over the course of 30 minutes, with treatment repeated as necessary, for example at intervals of days to weeks. It will be appreciated that appropriate dosages of the modified T cells, and compositions comprising the modified T cells, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular cells, the route of administration, the time of administration, the rate of loss or inactivation of the cells, the duration of the treatment, other drugs, compounds, and/or materials used in combination, and the age, sex, weight, condition, general health, and prior medical history of the patient. The amount of cells and the route of administration will ultimately be at the discretion of the physician, although generally the dosage will be to achieve local concentrations at the site of action which achieve the desired effect without causing substantial harmful or deleterious side-effects.

While the modified T cells may be administered alone, in some circumstances the modified T cells may be administered cells in combination with the target antigen, APCs displaying the target antigen, and/or IL-2 to promote expansion in vivo of the population of modified T cells. The population of modified T cells may be administered in combination with one or more other therapies, such as cytokines e.g. IL-2, cytotoxic chemotherapy, radiation and immuno-oncology agents, including checkpoint inhibitors, such as anti-B7-H3, anti-B7-H4, anti-TIM3, anti-KIR, anti-LAG3, anti-PD-1, anti-PD-L1, and anti-CTLA4 antibodies.

The one or more other therapies may be administered by any convenient means, preferably at a site which is separate from the site of administration of the modified T cells.

Administration of modified T cells can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician. Preferably, the modified T cells are administered in a single transfusion of a least 1×10⁹ T-cells.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Other aspects and embodiments of the invention provide the aspects and embodiments described above with the term “comprising” replaced by the term “consisting of” and the aspects and embodiments described above with the term “comprising” replaced by the term “consisting essentially of”.

Certain aspects and embodiments of the invention will now be illustrated by way of example and with reference to the figures described above.

### EXPERIMENTS

1. Methods

1.1 Design and Production of IL-7 expression constructs

A lentiviral expression vector was designed that permitted the inducible expression of IL-7 following recognition of the cognate peptide MHC1 complex. The inducible element was based on NFAT/AP1 transcriptional response element (TRE) that is present in the human IL2 promoter (Macian et al., 2001 Oncogene). The selective inducible expression of IL-7 is a result of TCR activation, and Ca2 dependent translocation of NFAT to the nucleus where it can promote transcription of genes which harbour an NFAT TRE.

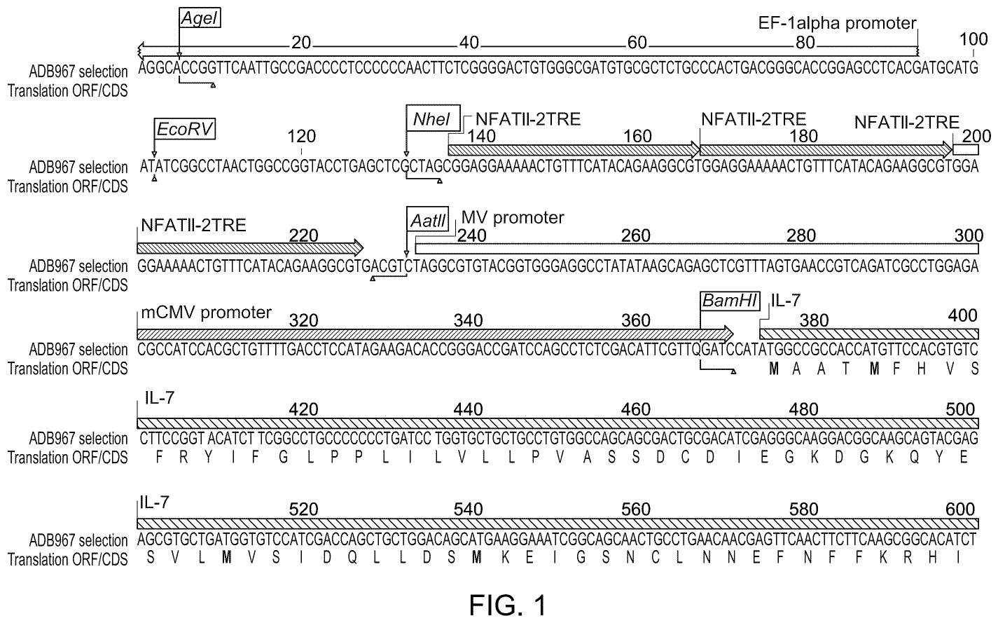

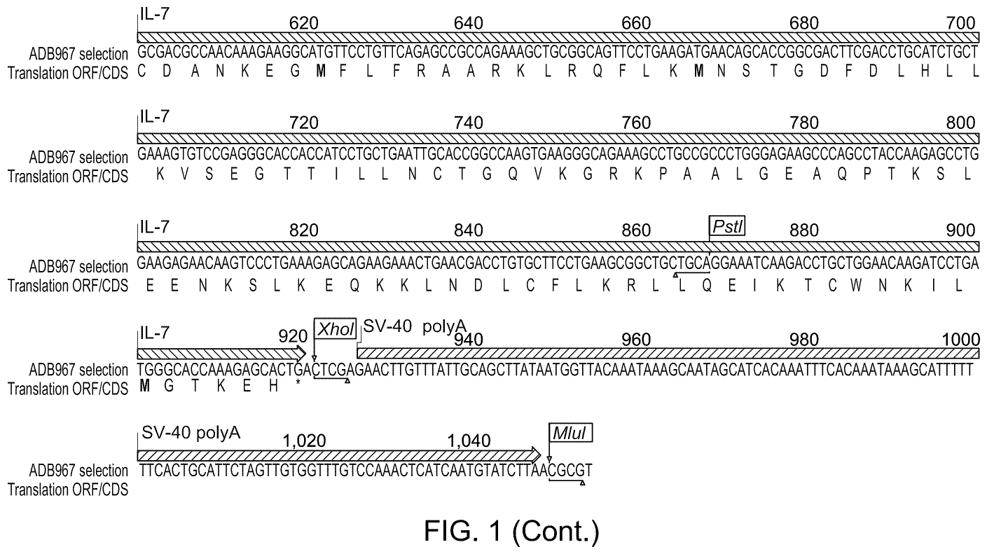

The IL-7 expression cassette was is composed of four elements as illustrated in FIG. 1.
1. 3 tandem repeats of the human IL2 promoter NFAT TRE highlighted in red (GGAGGAAAAACTGTTTCATACAGAAGGCGT)
2. Minimal CMV promoter (green)
3. Kozak sequence (GCCGCCACCATG) and codon optimised IL-7 coding sequence (Yellow). Supplementary FIG. 1 contains the wild-type nucleotide sequence
4. SV40 polyadenylation signal (purple)

The sequence upstream of the EcorV restriction (highlighted in green in FIG. 1) corresponds to the lentiviral vector backbone sequence and was included in the synthetic construct in order to maintain the coding sequence of the EF1A promoter. Additional unique restriction sites were also designed into the synthetic construct in order to permit further engineering of the construct should it be required. These include:
1 EcoRV (GATATC) and NSI-I (ATGCAT) restriction sites upstream of the NFAT IL-2 TRE
2. NdeI restriction site CATATG immediately upstream of the IL-7 CDS
3. XhoI (CTCGAG) immediately following the TGA stop codon in the IL-7 CDS
4. AatII restriction site (GACGTC) immediately upstream of the mCMV promoter.

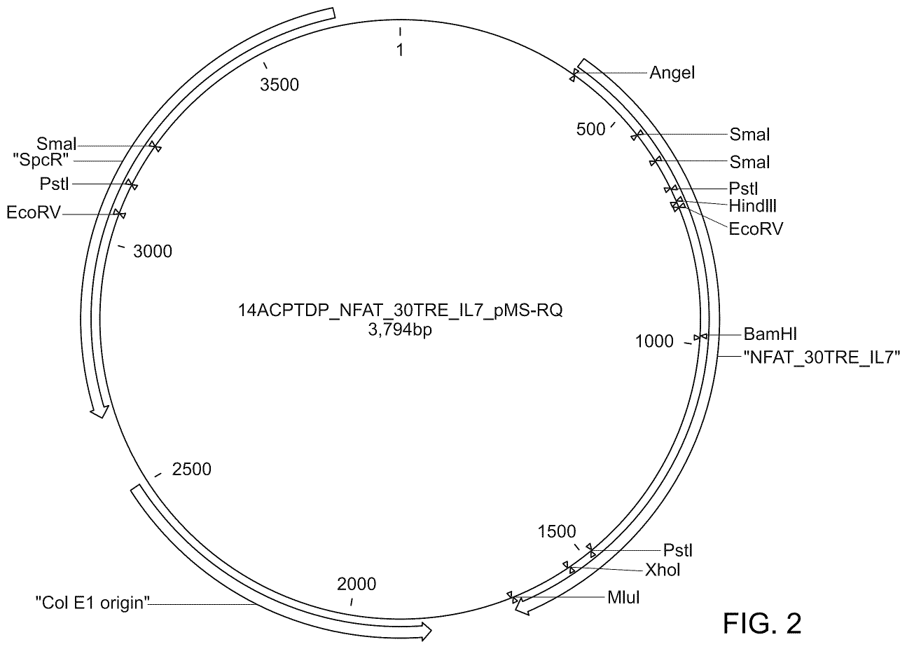

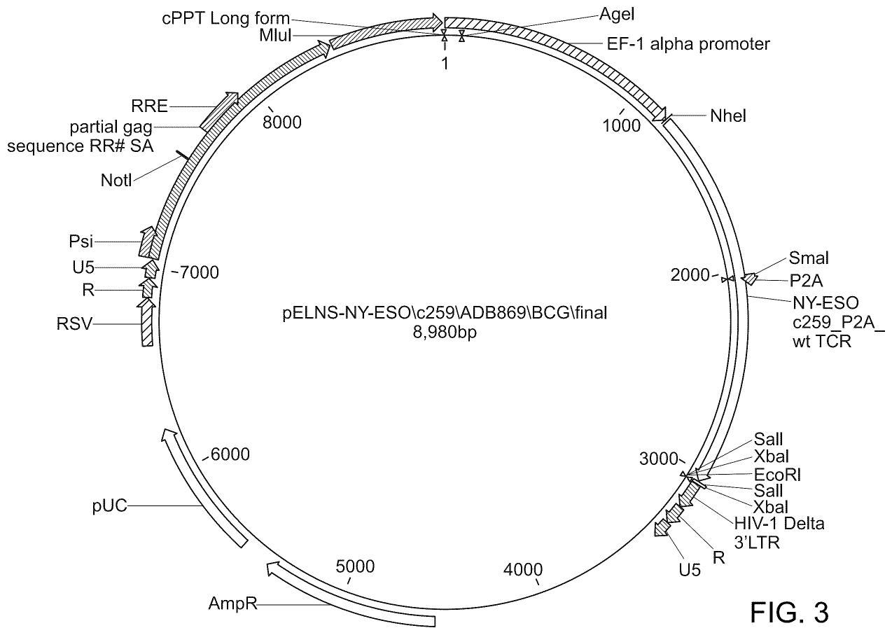

This construct was designed to be cloned between the AgeI and MluI restriction sites within a construct expressing a TCR alone (FIG. 2) and will permit transcription of the IL-7 from the antisense strand and in reverse orientation to the SPEAR TCR. Additional constructs were designed that permitted the constitutive expression of IL-7 and the SPEAR TCR. These constructs encode IL-7 and the TCRα and TCRβ chains as a single open reading frame. The individual polypeptides are separated by self-cleaving 2A-like peptides that permit ribosomal skipping and the expression of multiple polypeptides from a single transcript.

### 1.2 Generation of ADB967 NFAT IL-7 TCR1

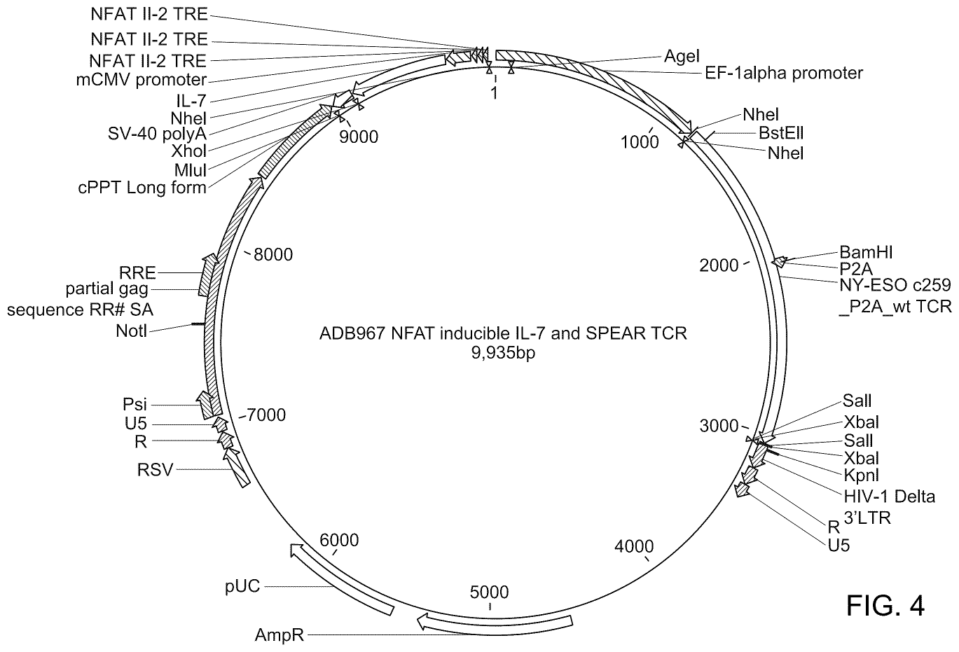

The NFAT IL-7 cassette (SEQ ID NO: 1) was synthesised synthetically by GeneArt (Thermo Fisher Scientific) with 5′ AgeI (ACCGGT) 3′ MluI (ACGCGT) restriction sites and was provided in a standard cloning vector (pMS-RQ) (FIG. 2). The NFAT_IL-7 containing vectors and ADB934 (TCR1) were digested with AgeI and MluI and the fragments of interest were purified by gel extraction using a NucleoSpin® gel extraction kit according to manufacturer's instruction. Digested fragments were ligated using T4 DNA ligase (NEB) according to standard protocols. Clones were screened by restriction enzyme digest and verified by sequencing (see the vector map of FIG. 4).

### 1.3 Generation of ADB1099 Constitutive IL-7_TCR1

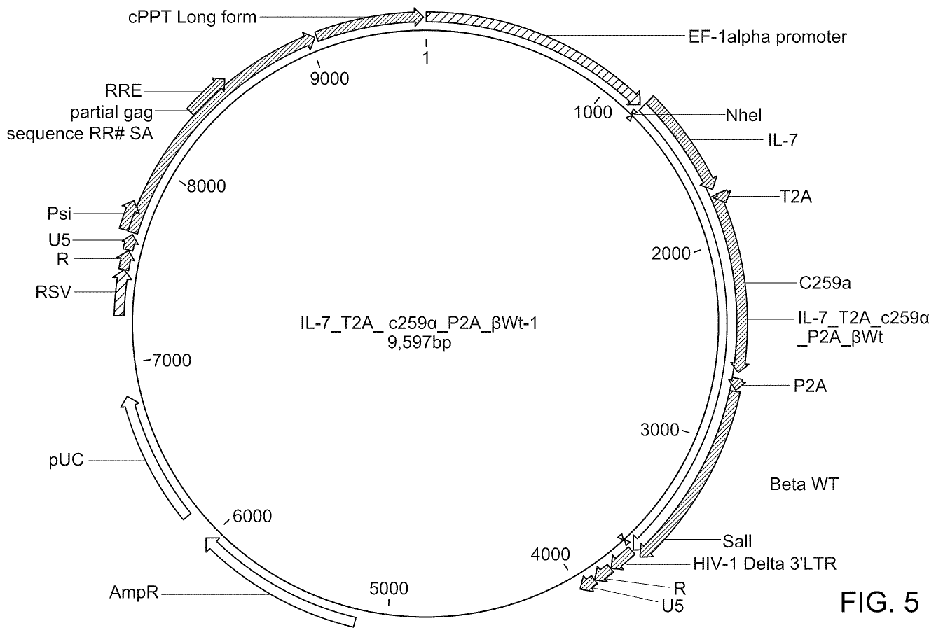

The IL-7_T2A_TCR1 insert was generated by overlapping PCR. The IL-7 CDS was amplified with primers NHEI_IL_7F and IL7_T2AR. The TCR1 CDS was amplified with the primers T2AF and BETA_SAL_REVIII. The TCR1 CDS was amplified from an existing in-house construct ADB951. PCR reactions were performed with Q5 DNA polymerase (NEB) according to standard protocols and PCR products were purified by gel extraction using a NucleoSpin® gel extraction kit according to manufacturer's instruction. The two fragments were fused together by overlapping PCR with the NHEI_IL_7F and BETA_SAL_REVIII primers which contain NheI (GCTAGC) and SalI (GTCGAC) restriction sites (underlined). Digested fragments were ligated using T4 DNA ligase (NEB) according to standard protocols. Clones were screened by restriction enzyme digest and verified by sequencing (see the vector map of FIG. 5).

| Primer | Sequence |
|---|---|
| NHEI_IL_7F | TAATGCTAGCGCCGCCACCATGTTCCACG |
| IL_7T2AR | CAGCAGGCTGCCTCTGCCCTCGCCAGAGC |
| | CGCTTCTCTTGGCTCTGCTTCCGTGCTCT |
| | TTGGTGCCCATCAGG |
| T2AF | AGGGCAGAGGCAGCCTGCTG |
| BETA_SAL_ | ATTATTGTCGACTTAGCCCCGGCTGTCCT |
| REVIII | TCCGCTTCACC |

### 1.4 Generation of ADB1581 NFAT IL-7 TCR2

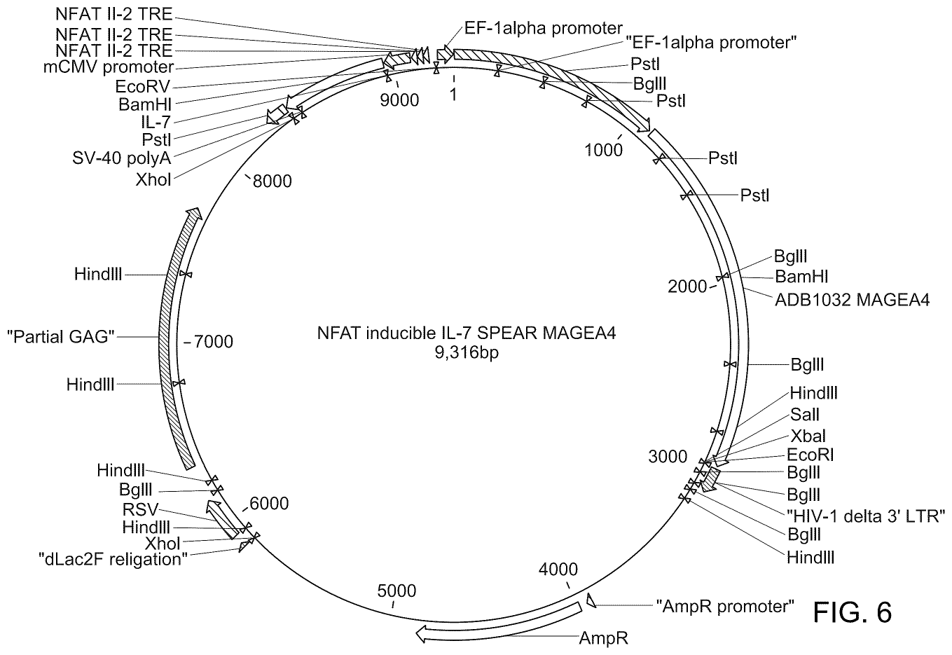

The TCR2 insert was subcloned from in-house construct ADB1535 into ADB1224. ABD1535 and ADB1224 were digested with NheI and SalI restriction enzymes and fragments of interest were purified by gel extraction using a NucleoSpin® gel extraction kit according to manufacturer's instruction. Digested fragments were ligated using T4 DNA ligase (NEB) according to standard protocols. The NFAT IL-7 cassette was subcloned from ADB967 (NFAT-IL-7_TCR1) into this new TCR2 lentiviral expression vector between the AgeI and MluI restriction sites. Clones were screened by restriction enzyme digest and verified by sequencing (see the vector map of FIG. 6).

### 1.5 Generation of ADB1580 Constitutive IL-7_F2A TCR2

The IL-7 CDS was PCR amplified from ABD1099 with the following primers

FWD-
5′ TAATGCTAGCGCCGCCACCATGTTCCACGTGTCC 3′
and
Rev
5′-CTTCACGGGCGCGCCAGAGCCGCTTCTCTTGG- 3′.

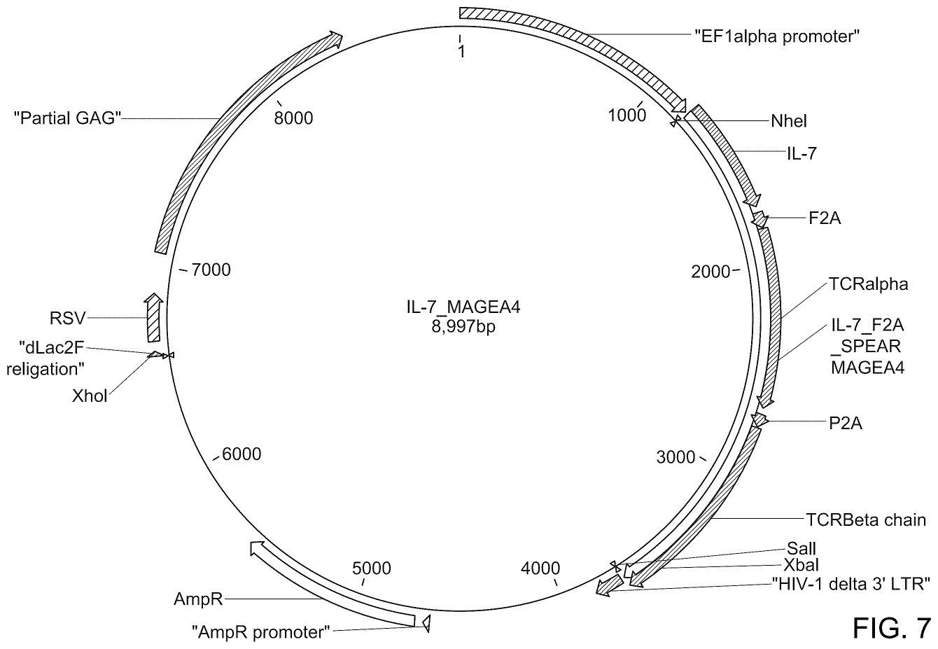

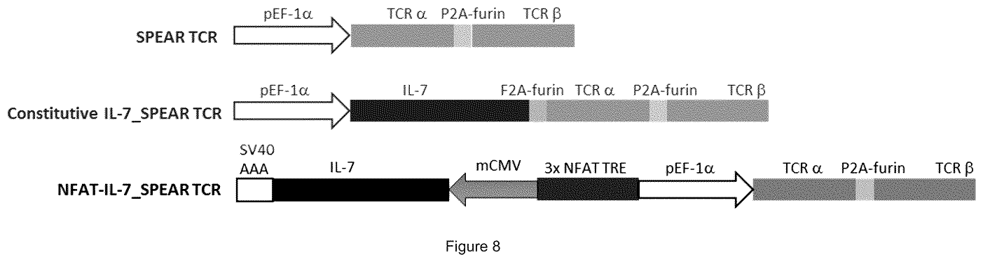

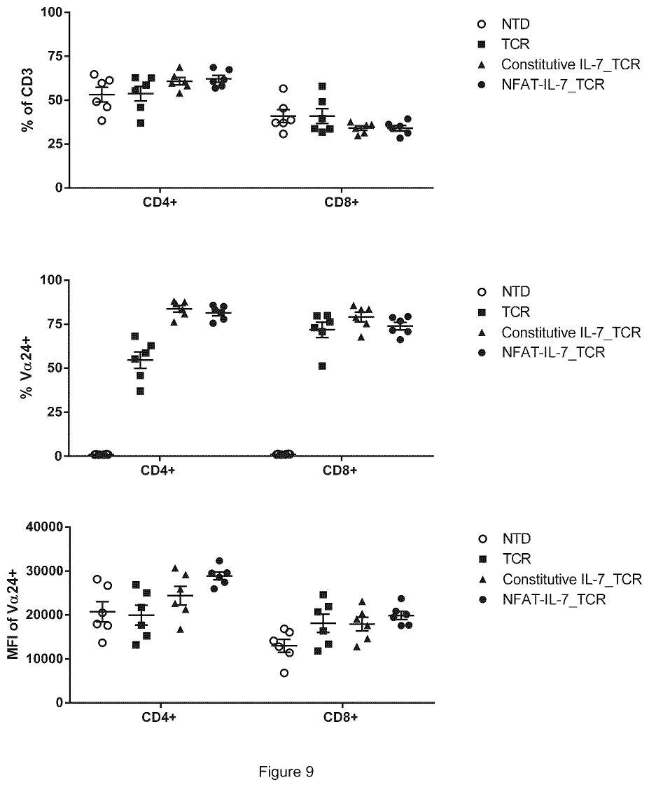

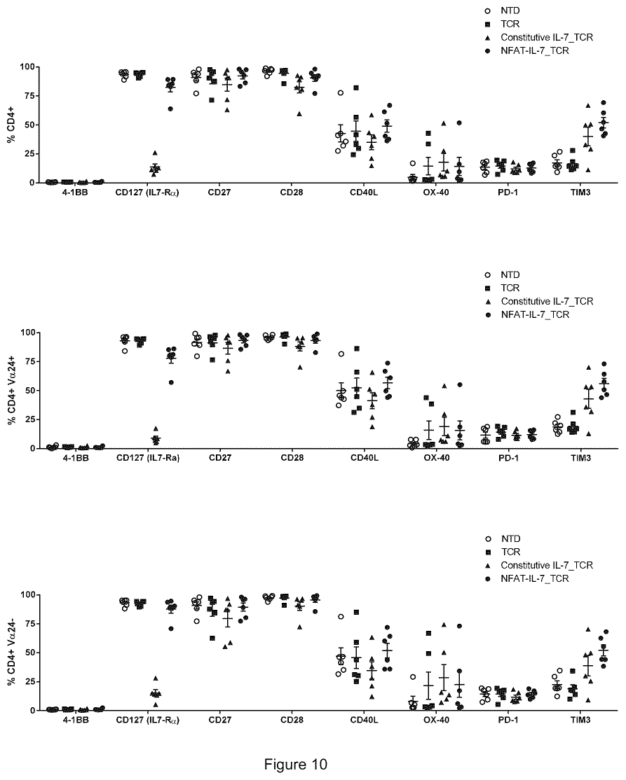

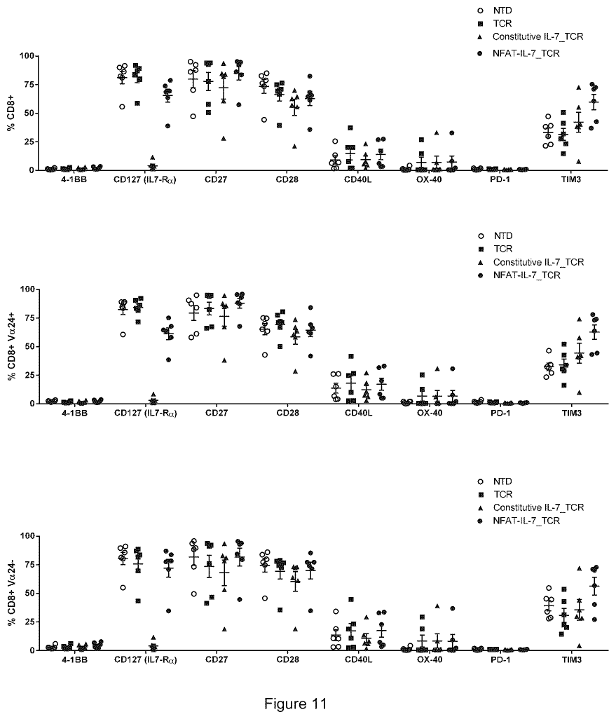

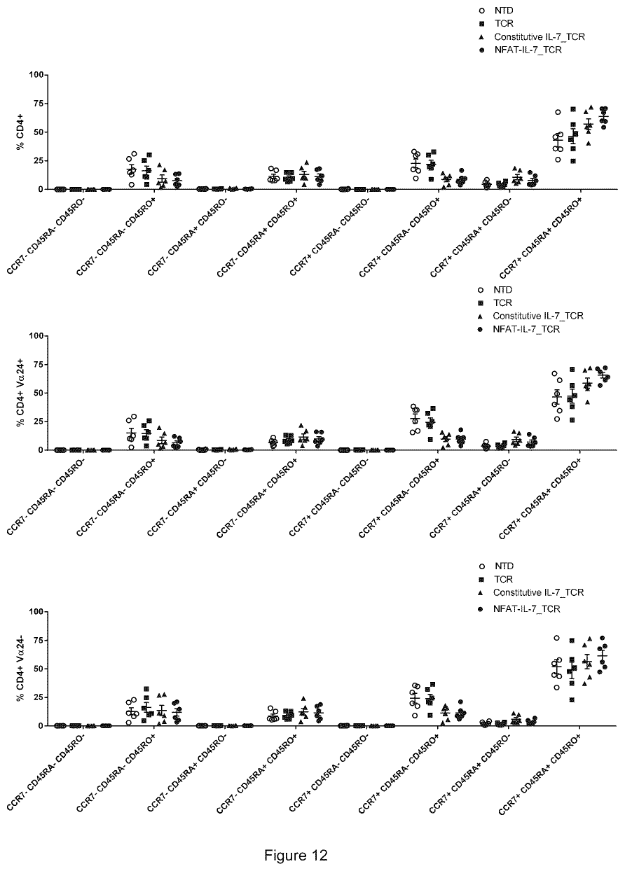

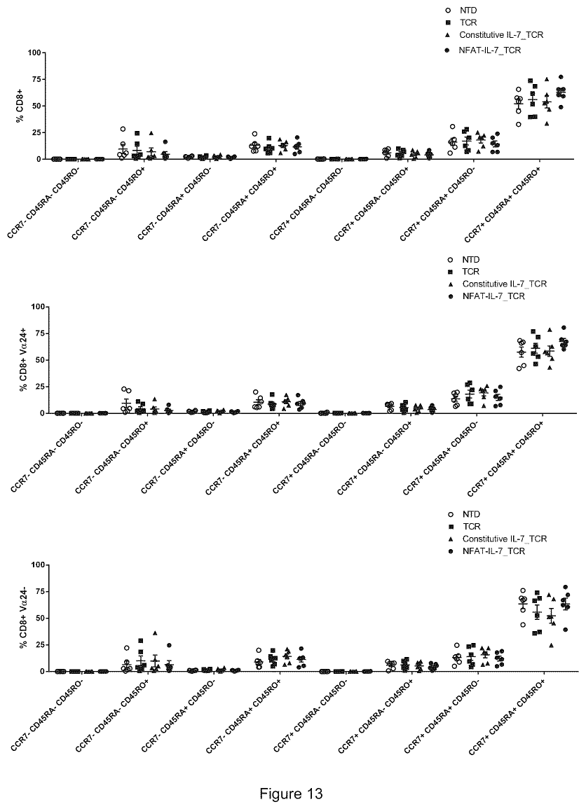

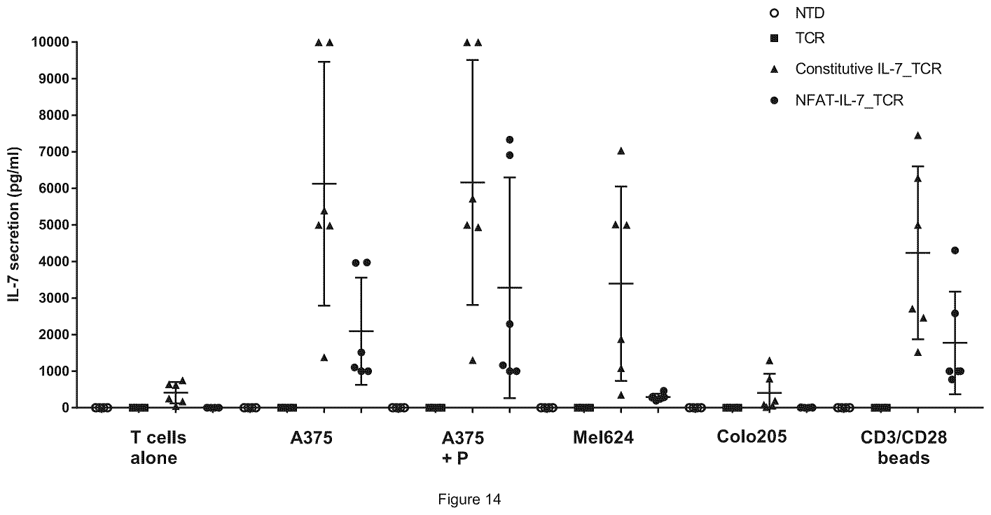

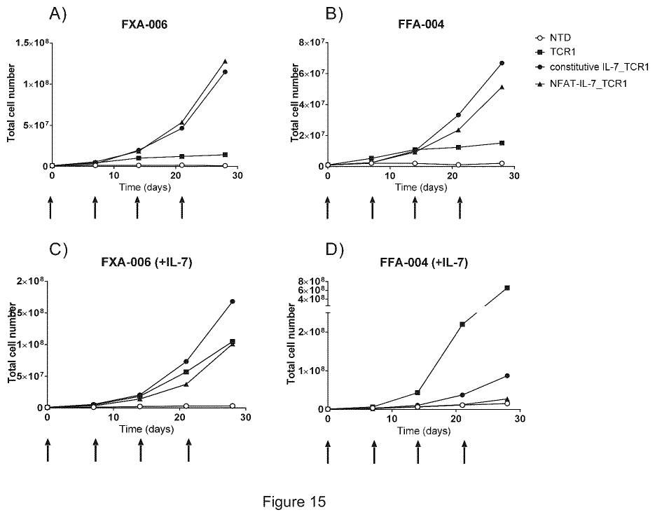

These include NheI (GCTAGC) and AscI (GGCGCGCC) restriction sites (underlined). PCR products were purified by gel extraction and purified by gel extraction using a NucleoSpin® gel extraction kit according to manufacturer's instruction. The PCR product and (ADB1488) were digested with NheI and AscI and fragments of interest purified by gel extraction using a NucleoSpin® gel extraction kit according to manufacturer's protocol. Digested fragments were ligated using T4 DNA ligase (NEB) according to standard protocols. Clones were screened by restriction enzyme digest and verified by sequencing (see the vector map of FIG. 7).

### 1.6 T Cell Production

PBLs from six donors were separated from whole blood with additional CD14 depletion (PBL) using MACS isolation kits and associated manufacturer's protocols. T cells were transduced with different lentiviral constructs including: TCR and NFAT_IL-7 TCR (see FIG. 1). T cell transductions were performed in the presence of 1 mg/ml F108 Poloxamer. The F108 Poloxamer had previously been dissolved in water to make a stock solution at 100 mg/ml and was then sterilised using a 0.2 μm filter before storing at 4° C. 1 mg/ml F108 Poloxamer was added to each well of cells (including NTD cells) at the same time as addition of the lentiviral particles.

Transduced cells were expanded for 14 days. T cells were fed with 100 U/ml Proleukin on day 10 but fed with fresh media without Proleukin on day 12 to allow the cells more time to rest prior to use for assays, particularly for proliferation assays.

On day 14 cells were frozen. Non-transduced (NTD) T cells were produced at the same time as the transduced T cells but fresh virus medium (R10+Hepes) was added to the stimulated T cells in place of lentiviral particles.

### 1.7 Cytokine Secretion Assays for ELISA

Cytokine production assays were typically carried out as follows. On the day of the assay, A375, Mel624 and Colo205 cells were harvested, resuspended in R10 and counted. Some of the A375 cells were pulsed for 2 hours at 37° C./5% CO₂ with 10 μM MAGE-A4 peptide, before washing three times. Target cells were typically plated out at 50,000 cells/well in a volume of 100 μl into 96 well flat bottom plates in triplicate wells. T cells were thawed, washed, resuspended in R10 at no less than 1×10⁶ cells/ml and rested for 1-2 h at 37° C./5% CO₂. Non-transduced and transduced effector cells were counted and were plated at 120,000 cells per well in 100 μl assay medium, in triplicate wells, to give a final volume of 200 μl. Note: to account for differences in transduction between the different constructs, the cells were normalised by adding NTD T cells from the same donor to give equivalent transduction efficiencies. Plates were then put in the incubator at 37° C./5% CO₂ for 48 hours. After 48 hours, the assay plates were centrifuged and supernatant transferred to new 96 well plates, which were stored at −20° C. until analysis by ELISA which was performed at a later date.

### 1.8 Il-7 ELISA

This assay was performed in 96 well half area plates using the Duoset Human IL-7 ELISA kit according to the manufacturer's protocol with modifications. Briefly, as the 96 well half area plates were used, 25 μl/well (or 50 μl of blocking reagent) was used for all reagents instead of 100 μl as indicated in the protocol. In most cases the samples were used undiluted. If they were diluted they were diluted in assay medium. The assays were developed using commercial TMB substrate solution for approx. 10 minutes and the reaction stopped with IN H2SO4. Assays were read using the Spectrastar Omega at 450 nm with wavelength correction set to 540 nm. Data were analysed using Spectrastar data analysis software. Data points greater than the top concentration on the standard curve were normally allocated the value of the top concentration. For most assays the standard curve was extended to provide a greater range than indicated by the manufacturer, with a top concentration of 1000 μg/ml. Samples and standard curves were either performed using duplicate or triplicate wells and the curve fitting was performed using four parameter logistic curve fitting where possible.

### 1.9 T Cell Phenotyping

On the day of the assay, the T cells were thawed, washed twice in PBS, counted and adjusted to 1×10⁶ cells/ml in PBS. Flow cytometry was performed on T cells. The list of antibodies used can be found in Tables 1 and 2.

TABLE 1
List of antibodies and volumes used to
identify proliferating T cells at the end
of the expansion phase (Panel 1)

| | Volume/ | |
|---|---|---|
| Antibody | test (μl) | |
| CD45RA-PE Cy7 | 1.25 | μl |
| (BioLgend: 304126) | | |
| CD45RO-PerCP Cy5.5 | 1.25 | μl |
| (BioLegend: 304222) | | |
| CCR7-PE CF594 | 5 | μl |
| (BD Bioscience: 562381) | | |
| Vα24-PE | 20 | |
| (Beckman Coulter: IM2283) | | |
| PD-1-BV786 | 2.5 | μl |
| (BioLegend: 329929) | | |
| CD4-BV650 | 2.5 | μl |
| (BD Bioscience: 56875) | | |
| CD8-APC-ef780 | 2.5 | μl |
| (eBioscience: 47-0087-42) | | |
| CD3-FITC | 2.5 | μl |
| (eBioscience: 11-0036-42) | | |
| TIM-3-APC | 5 | μl |
| (R&D Systems: FAB2365A) | | |
| IL7Rα (CD127)-BV421 | 5 | μl |
| (BioLegend: 47-1278-42) | | |

| | |
|---|---|
| LIVE/DEAD ® Fixable | 1/800 |
| Aqua Dead Cell Stain | dilution |
| (Life Technologies: L34957) | |

TABLE 2
List of antibodies and volumes used to
identify proliferating T cells at the end
of the expansion phase (Panel 2)

| | Volume/ | |
|---|---|---|
| Antibody | test (μl) | |
| CD8-APC-ef780 | 2.5 | μl |
| (eBioscience: 47-0087-42) | | |
| CD4-BV650 | 2.5 | μl |
| (BD Bioscience: 56875) | | |
| CD45RA-PE Cy7 | 1.25 | μl |
| (BioLgend: 304126) | | |
| CCR7-PE CF594 | 5 | μl |
| (BD Bioscience: 562381) | | |
| Vα24-PE | 20 | |
| (Beckman Coulter: IM2283) | | |
| OX-40(CD134)-FITC | 5 | μl |
| (BD Bioscience: 555837) | | |
| CD40L (CD154)-BV421 | 5 | μl |
| (BD Bioscience: 563886) | | |
| CD28-PerCPCy5.5 | 2.5 | μl |
| (BD Bioscience: 560685) | | |
| CD27-BV786 | 5 | μl |
| (BD Bioscience: 563327) | | |
| 4-1BB-APC | 2.5 | μl |
| (BD Bioscience:550890) | | |

| | |
|---|---|
| LIVE/DEAD ® Fixable | 1/800 |
| Aqua Dead Cell Stain | dilution |
| (Life Technologies: L34957) | |

### 1.10 Restimulation Assay

The restimulation assays were carried out as follows. On the first day of the assay (day 0) T cells were thawed, washed, resuspended in R10 at no less than 1×10⁶ cells/ml and rested.

Cells were counted and in order to account for differences in transduction between the different lentiviral preparations, the cells were normalised by adding NTD T cells from the same donor to give equivalent transduction efficiencies within a batch of donor T cells.

T cells were plated out in R10 at 1×10⁶/ml, with 1 ml of T cells per well in a 24 well plate. A375 target cells were harvested and then irradiated (48 Gy). A375 cells were resuspended at approximately 1×10⁶/ml and 1 ml per well added to the wells containing T cells. This gave a final volume of 2 ml/well with 1:1 T cells and irradiated A375 cells. In control wells recombinant human IL-7 was added to the cells at a final concentration of 20 ng/ml. Co-cultures of T cells and irradiated A375 cells with or without exogenous IL-7 were then cultured for 7 days at 37° C./5% CO₂.

On days 7, 14, 21 and 28, T cells were harvested and counted using semi-automated counting with Trypan Blue positive cells (dead cells) excluded. For wells containing T cells that did not express the TCR1, not all the Trypan Blue-negative (live) cells were T cells, as they were much larger than T cells. These were most likely to be irradiated A375 targets that were not yet positive for Trypan Blue staining. As many as possible of the larger, obviously non-T cells (A375) were manually excluded from the counts but live T cell counts from those wells (containing NTD) are likely to be an overestimate as they still contained some “apparently” live A375 targets. This was also reflected by the larger average size of the cells from those samples as A375 cells are larger than T cells. However in wells containing T cells expressing the TCR1 there were few or no large, live A375 cells remaining, so absolute counts and cell sizes are likely to be more accurate. The total live T cell count was then calculated.

Once the total number of live T cells had been calculated, T cells were split as follows. If the T cells had proliferated, they were diluted to a concentration of 1×10⁶/ml (to the nearest ml) by adding fresh R10 to the existing volume of T cells (in “old” R10) to the correct final concentration. “Old” R10 is defined as the media that the cells had been cultured in for the previous week. If however the T cells had contracted (i.e. the number of live T cells had reduced compared with the concentration originally plated out), cells were centrifuged (1500 rpm, 5 minutes) and then resuspended in the appropriate volume of “old” R10.

T cells were plated out at 1 ml/well (˜1×10⁶ T cells/ml) in 24 well plates. Where possible, all the T cells were plated out as they expanded, so that the total number of T cells presented in the figures was actually the total number of T cells produced. In some instances, particularly at later time points as the number of T cells grew increasingly large (usually greater than 8-12×10⁶ cells), only 8-12×10⁶ T cells were plated out for restimulation. The total number of T cells for those samples at subsequent time-points was then re-calculated by factoring in the number of T cells that had been originally plated out at the previous time-point. For example, if we had counted 20×10⁶ T cells on day 21, we would only plate out 8×10⁶ of those cells on day 21 for restimulation. The total cell numbers on day 28, which would have been generated if we had plated out all 20×10⁶ cells, would be re-calculated as follows:
1. Fold change in cell numbers=Cell count after restimulation (on day 28)/Cell number plated out for restimulation (8×10⁶ originally plated out on day 21)
2. Re-calculated total cell numbers on day 28=Fold change in cell numbers (between days 21-28)×total number of cells that were available for plating out on day 21 (e.g. 20×10⁶)

A375 target cells were harvested and irradiated at 48 Gy to prevent their proliferation, and then added to the T cells at approximately 1×10⁶/ml, 1 ml/well (1:1 T cell: A375) in R10. In control wells recombinant human IL-7 was added to the cells at a final concentration of 20 μg/ml (assuming that all the IL-7 had been “consumed” in the well). This restimulation process was repeated on days 7, 14 and 21.

### 1.11 In Vivo Studies

PBL from a Leukopak were depleted of CD14 using the methods previously described. T cells were transduced as previously described and expanded for 14 days before freezing. In addition to the NTD T cells and T cells transduced with constructs expressing TCR1, NFAT-IL-7 TCR1 or constitutive IL-7 TCR1, T cells were also transduced with a construct expressing NFAT-IL-7_irrelevant TCR.

On day 0, immunodeficient CIEA NOG (NOD.Cg-Prkdc_{scid} Il2g_{tmlSug}/JicTac) female mice aged 6-8 weeks were injected intravenously (i.v.) with 1×10⁶ Mel624 tumour cells that had previously been transduced with a lentiviral construct expressing GFP/Luciferase. On day 6 post-tumour cell implantation, mice were imaged and randomised into 8 groups of 8 mice groups, based on luciferase signal strength so that each group had an equal mean total flux. On day 7, T cells were thawed and the frequency of transduced T cells between the different constructs was normalised using NTD T cells to ˜42% TCR VB+/CD3+. Mice were then i.v. injected either with nothing (tumour only control) or with 2×10⁶ total T cells (NTD, TCR1, NFAT-IL-7_TCR1, Constitutive IL-7_TCR1, NFAT-IL-7_control TCR).

Animals were imaged using the Bruker In Vivo Xtreme imaging system on day 6 and then once weekly to measure disease burden and follow disease progression. At later stages in the study animals were imaged twice weekly. Prior to imaging, animals were injected i.p. with 150 mg/kg of luciferin (5 ml/kg) and anaesthetised with isoflurane. After the exposure an X-ray picture was taken to aid orientation and organ positioning. At the time of analysis, images were converted to photons/second/mm² (P/s/mm²) which allows comparison of images acquired using different exposure times, the scale was adjusted and the bioluminescence image was superimposed upon the X-ray image. A region of interest was set to measure the bioluminescence signal from the whole animal. The size of the ROI was kept identical for all images. Mice were also weighed at least 3 times per week and were culled when either weight loss or poor condition indicated.

### Results

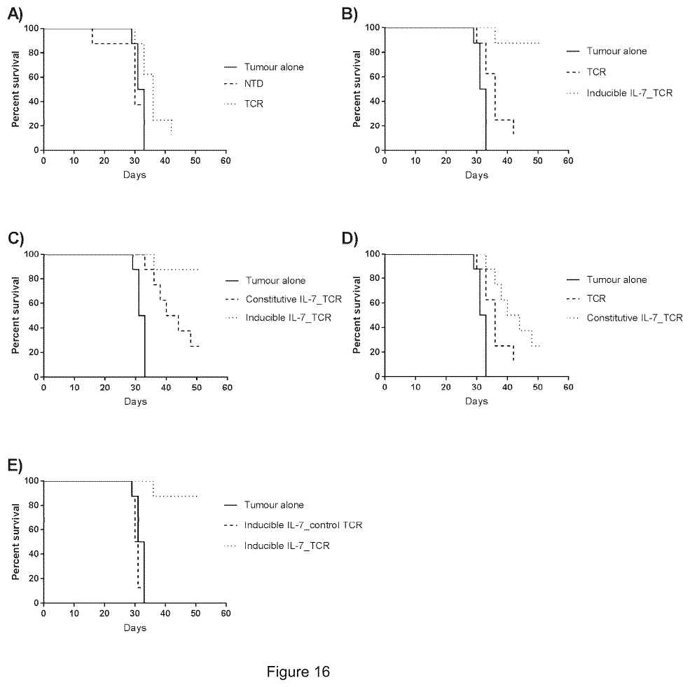

Mice injected with T cells co-expressing inducible IL-7 with an engineered TCR were found to show improved survival compared with cells expressing TCR alone or TCR together with constitutive IL-7 (FIG. 16).

```
Sequences:
ACCGGTTCAATTGCCGACCCCTCCCCCCAACTTCTCGGGGACTGTGGGCGATGTGCGCTCTGCCCACTGACGGGC
ACCGGAGCCTCACGATGCATGATATCGGCCTAACTGGCCGGTACCTGAGCTCGCTAGCGGAGGAAAAACTGTTTC
ATACAGAAGGCGTGGAGGAAAAACTGTTTCATACAGAAGGCGTGGAGGAAAAACTGTTTCATACAGAAGGCGTga
cgtcTAGGCGTGTACGGTGGGAGGCCTATATAAGCAGAGCTCGTTTAGTGAACCGTCAGATCGCCTGGAGACGCC
ATCCACGCTGTTTTGACCTCCATAGAAGACACCGGGACCGATCCAGCCTCTCGACATTCGTTGGATCCATATGGC
CGCCACCATGTTCCATGTTTCTTTTAGGTATATCTTTGGACTTCCTCCCCTGATCCTTGTTCTGTTGCCAGTAGC
ATCATCTGATTGTGATATTGAAGGTAAAGATGGCAAACAATATGAGAGTGTTCTAATGGTCAGCATCGATCAATT
ATTGGACAGCATGAAAGAAATTGGTAGCAATTGCCTGAATAATGAATTTAACTTTTTTAAAAGACATATCTGTGA
TGCTAATAAGGAAGGTATGTTTTTATTCCGTGCTGCTCGCAAGTTGAGGCAATTTCTTAAAATGAATAGCACTGG
TGATTTTGATCTCCACTTATTAAAAGTTTCAGAAGGCACAACAATACTGTTGAACTGCACTGGCCAGGTTAAAGG
AAGAAAACCAGCTGCCCTGGGTGAAGCCCAACCAACAAAGAGTTTGGAAGAAAATAAATCTTTAAAGGAACAGAA
AAAACTGAATGACTTGTGTTTCCTAAAGAGACTATTACAAGAGATAAAAACTTGTTGGAATAAAATTTTGATGGG

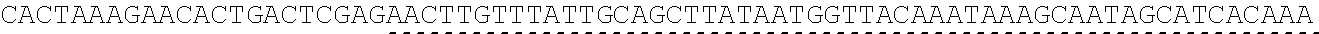

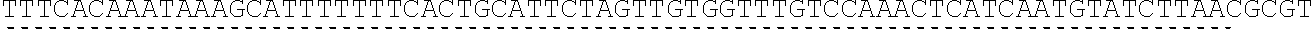


SEQ ID NO: 1 Nucleotide sequence of the NFAT IL-7 inducible cassette.
Key:
Full underline = Tandem repeats of the human IL2 promoter NFAT TRE
(GGAGGAAAAACTGTTTCATACAGAAGGCGT).
Dashed underline = Minimal CMV promoter.
No underline = The Kozak sequence (GCCGCCACCATG) and codon optimised IL-7 coding
sequence.
Dotted underline = The SV40 polyadenylation signal.
atgttccatgtttcttttaggtatatctttggacttcctcccctgatccttgttctgttg
  M F H V S F R Y I F G L P P L I L V L L
ccagcagcatcatctgattgtgatattgaaggtaaagatggcaaacaatatgagagtgtt
  P V A S S D C D I E G K D G K Q Y E S V
ctaatggtcagcatcgatcaattattggacagcatgaaagaaattggtagcaattgcctg
  L M V S I D Q L L D S M K E I G S N C L
aataatgaattaacttttttaaaagacatatctgtgatgctaataaggaaggtatgttt
  N N E F N P F K R H I C D A N K E G M F
ttattccgtgctgctcgcaagttgaggcaatctcttaaaatgaatagcactggtgacttt
  L F R A A R K L R Q F L E M N S T G D F
gatctccacttattaaaagtttcagaaggcacaacaatactgttgaactgcactggccag
  D L E L L K U S E G T T I L L N C T G Q
gttaaaggaagaaaaccagctgccctgggtgaagcccaaccaacaaagagtttggaagaa
  V K G R K P A A L G K A Q P I K S L E K
aataaatctttaaaggaacagaaaaaactgaatgacttgtgtttcctaaagagactatta
  N K S L K E Q E K L N D L C F L H A L L
caagagataaaaacttgttggaataaaatcttgatgggcactaaagaacactga
  Q E I K T C N N K I L M G T K E H —
Translated nucleotide sequence (SEQ ID NO: 2) and protein sequence (SEQ ID NO: 3) of codon
optimised IL-7
MFHVSFRYIFGLPPLILVLLPVASSDCDIEGKDGKQYESVLMVSIDQLLDSMKEIGSNCLNNEFNFFK
RHICDANKEGMFLFRAAPKLRQFLKMNSTGDFDLHLLKVSEGTTILLNCTGQVKGRKPAALGEAQTPK
SLEENKSLKEQKKLNDLCFLKRLLQEIKTCWNKILMGTKEH*
SEQ ID NO: 3 IL-7 protein sequence
ATGGAGACCCTGCTGGGCCTGCTGATCCTGTGGCTGCAGCTCCAGTGGGTGTCCAGCAAGCAGGAGGT
GACCCAGATCCCTGCCGCCCTGAGCGTGCCCGAGGGCGAGAACCTGGTGCTGAACTGCAGCTTCACCG
ACTCCGCCATCTACAACCTGCAGTGGTTCCGGCAGGACCCCGGCAAGGGCCTGACCAGCCTGCTGCTG
ATCCAGAGCAGCCAGCGGGAGCAGACCAGCGGACGGCTGAACGCCAGCCTGGACAAGAGCAGCGGCCG
GAGCACCCTGTACATCGCCGCCAGCCAGCCCGGCGACAGCGCCACCTACCTGTGCGCTGTGCGGCCTC
TGTACGGCGGCAGCTACATCCCCACCTTCGGCAGAGGCACCAGCCTGATCGTGCACCCCTACATCCAG
AACCCCGACCCCGCCGTGTACCAGCTGCGGGACAGCAAGAGCAGCGACAAGTCTGTGTGCCTGTTCAC
CGACTTCGACAGCCAGACCAATGTGAGCCAGAGCAAGGACAGCGACGTGTACATCACCGACAAGACCG
TGCTGGACATGCGGAGCATGGACTTCAAGAGCAACAGCGCCGTGGCCTGGAGCAACAAGAGCGACTTC
GCCTGCGCCAACGCCTTCAACAACAGCATTATCCCCGAGGACACCTTCTTCCCCAGCCCCGAGAGCAG
CTGCGACGTGAAACTGGTGGAGAAGAGCTTCGAGACCGACACCAACCTGAACTTCCAGAACCTGAGCG
TGATCGGCTTCAGAATCCTGCTGCTGAAGGTGGCCGGATTCAACCTGCTGATGACCCTGCGGCTGTGG
AGCAGCGGCTCCCGGGCCAAGAGAAGCGGATCCGGCGCCACCAACTTCAGCCTGCTGAAGCAGGCCGG
AGACGTGGAAGAAAACCCTGGCCCTAGGATGAGCATCGGCCTGCTGTGCTGCGCCGCCCTGAGCCTGC
TGTGGGCAGGACCCGTGAACGCCGGAGTGACCCAGACCCCCAAGTTCCAGGTGCTGAAAACCGGCCAG
AGCATGACCCTGCAGTGCGCCCAGGACATGAACCACGAGTACATGAGCTGGTATCGGCAGGACCCCGG
CATGGGCCTGCGGCTGATCCACTACTCTGTGGGAGCCGGAATCACCGACCAGGGCGAGGTGCCCAACG
GCTACAATGTGAGCCGGAGCACCACCGAGGACTTCCCCCTGCGGCTGCTGAGCGCTGCCCCCAGCCAG
ACCAGCGTGTACTTCTGCGCCAGCAGCTATGTGGGCAACACCGGCGAGCTGTTCTTCGGCGAGGGCTC
CAGGCTGACCGTGCTGGAGGACCTGAAGAACGTGTTCCCCCCCGAGGTGGCCGTGTTCGAGCCCAGCG
AGGCCGAGATCAGCCACACCCAGAAGGCCACACTGGTGTGTCTGGCCACCGGCTTCTACCCCGACCAC
GTGGAGCTGTCCTGGTGGGTGAACGGCAAGGAGGTGCACAGCGGCGTGTCTACCGACCCCCAGCCCCT
GAAGGAGCAGCCCGCCCTGAACGACAGCCGGTACTGCCTGTCCTCCAGACTGAGAGTGAGCGCCACCT
TCTGGCAGAACCCCCGGAACCACTTCCGGTGCCAGGTGCAGTTCTACGGCCTGAGCGAGAACGACGAG
TGGACCCAGGACCGGGCCAAGCCCGTGACCCAGATTGTGAGCGCCGAGGCCTGGGGCAGGGCCGACTG
CGGCTTCACCAGCGAGAGCTACCAGCAGGGCGTGCTGAGCGCCACCATCCTGTACGAGATCCTGCTGG
GCAAGGCCACCCTGTACGCCGTGCTGGTGTCTGCCCTGGTGCTGATGGCTATGGTGAAGCGGAAGGAC
AGCCGGGGCTAAGTCGAC
SEQ ID NO: 4 Nucleotide sequence between the NheI and SalI sites
METLLGLLILWLQLQWVSSKQEVIQIPAALSVPEGENLVLNCSFTDSAIYNLQWFRQDPGKGLTSLLL
IQSSQREQTSGRLNASLDKSSGRSTLYIAASQPGDSATYLCAVRPLYGGSYIPTFGRGTSLIVHPYIQ
NPDPAVYQLRDSKSSDKSVCLFTDEDSQTNVSQSKDSDVYIIDKIVLDMRSMDFKSNSAVAWSNKSDF
ACANAFNNSIIPEDTFFPSFESSCDVKLVEKSFETDTNLNFQNLSVIGFRILLLKVAGFNLLMTLRLW
SSGSRAKRSGSCATNFSLLKQAGDVEENPGPRMSIGLLCCAALSLLWAGPVNAGVIQTPKFQVLKTGQ
SMTLQCAQDMNHEYMSWYRQDPGMGLRLIHYSVGAGIIDQGEVPNGYNVSRSTTEDFPLRLLSAAPSQ
TSVYFCASSYVGNTGELFFGEGSRLTVLEDLKNVFPPEVAVFEPSEAEISHTQKATLVCLAIGFYPDH
VELSWWVNGKEVHSGVSTDPQPLKEQPALNDSRYCLSSPLRVSATFWQNPRNHFRCQVQFYGLSENDE
WIQDRAKPVTQIVSAEAWGRADCGFTSESYQQGVLSATILYEILLGKATLYAVLVSALVLMAMVKRKD
SRG*V
SEQ ID NO: 5 Protein sequence of TCR1
METLLGLLILWLQLQWVSSKQEVIQIPAALSVPEGENLVLNCSFTDSAIYNLQWFRQDPGKGLTSLLL
IQSSQREQTSGRLNASLDKSSGRSTLYIAASQPGDSATYLCAVRPLYGGSYIPTFGRGTSLIVHPYIQ
NPDPAVYQLRDSKSSDKSVCLFTDEDSQTNVSQSKDSDVYIIDKIVLDMRSMDFKSNSAVAWSNKSDF
ACANAFNNSIIPEDTFFPSFESSCDVKLVEKSFETDTNLNFQNLSVIGFRILLLKVAGFNLLMTLRLW
SSGSRAKRSGSCATNFSLLKQAGDVEENPGPRMSIGLLCCAALSLLWAGPVNAGVIQTPKFQVLKTGQ
SMTLQCAQDMNHEYMSWYRQDPGMGLRLIHYSVGAGIIDQGEVPNGYNVSRSTTEDFPLRLLSAAPSQ
TSVYFCASSYVGNTGELFFGEGSRLTVLEDLKNVFPPEVAVFEPSEAEISHTQKATLVCLAIGFYPDH
VELSWWVNGKEVHSGVSTDPQPLKEQPALNDSRYCLSSPLRVSATFWQNPRNHFRCQVQFYGLSENDE
WIQDRAKPVTQIVSAEAWGRADCGFTSESYQQGVLSATILYEILLGKATLYAVLVSALVLMAMVKRKD
SRG
SEQ ID NO: 6 TCR1 CDS
ACGCGTTAAGATACATTGATGAGTTTGGACAAACCACAACTAGAATGCAGTGAAAAAAATGCTTTATT
TGTGAAATTTGTGATGCTATTGCTTTATTTGTAACCATTATAAGCTGCAATAAACAAGTTCTCGAGTC
AGTGCTCTTTGGTGCCCATCAGGATCTTGTTCCAGCAGGTCTTGATTTCCTGCAGCAGCCGCTTCAGG
AAGCACAGGTCGTTCAGTTTCTTCTGCTCTTTCAGGGACTTGTTCTCTTCCAGGCTCTTGGTAGGCTG
GGCTTCTCCCAGGGCGGCAGGCTTTCTGCCCTTCACTTGGCCGGTGCAATTCAGCAGGATGGTGGTGC
CCTCGGACACTTTCAGCAGATGCAGGTCGAAGTCGCCGGTGCTGTTCATCTTCAGGAACTGCCGCAGC
TTTCTGGCGGCTCTGAACAGGAACATGCCTTCTTTGTTGGCGTCGCAGATGTGCCGCTTGAAGAAGTT
GAACTCGTTGTTCAGGCAGTTGCTGCCGATTTCCTTCATGCTGTCCAGCAGCTGGTCGATGGACACCA
TCAGCACGCTCTCGTACTGCTTGCCGTCCTTGCCCTCGATGTCGCAGTCGCTGCTGGCCACAGGCAGC
AGCACCAGGATCAGGGGGGGCAGGCCGAAGATGTACCGGAAGGACACGTGGAACATGGTGGCGGCCAT
ATGGATCCAACGAATGTCGAGAGGCTGGATCGGTCCCGGTGTCTTCTATGGAGGTCAAAACAGCGTGG
ATGGCGTCTCCAGGCGATCTGACGGTTCACTAAACGAGCTCTGCTTATATAGGCCTCCCACCGTACAC
GCCTAGACGTCACGCCTTCTGTATGAAACAGTTTTTCCTCCACGCCTTCTGTATGAAACAGTTTTTCC
TCCACGCCTTCTGTATGAAACAGTTTTTCCTCCGCTAGCGAGCTCAGGTACCGGCCAGTTAGGCCGAT
ATCATGCATCGTGAGGCTCCGGTGCCCGTCAGTGGGCAGAGCGCACATCGCCCACAGTCCCCGAGAAG
TTGGGGGGAGGGGTCGGCAATTGAACCGGTGCCTAGAGAAGGTGGCGCGGGGTAAACTGGGAAAGTGA
TGTCGTGTACTGGCTCCGCCTTTTTCCCGAGGGTGGGGGAGAACCGTATATAAGTGCAGTAGTCGCCG
TGAACGTTCTTTTTCGCAACGGGTTTGCCGCCAGAACACAGGTAAGTGCCGTGTGTGGTTCCCGCGGG
CCTGGCCTCTTTACGGGTTATGGCCCTTGCGTGCCTTGAATTACTTCCACCTGGCTGCAGTACGTGAT
TCTTGATCCCGAGCTTCGGGTTGGAAGTGGGTGGGAGAGTTCGAGGCCTTGCGCTTAAGGAGCCCCTT
CGCCTCGTGCTTGAGTTGAGGCCTGGCCTGGGCGCTGGGGCCGCCGCGTGCGAATCTGGTGGCACCTT
CGCGCCTGTCTCGCTGCTTTCGATAAGTCTCTAGCCATTTAAAATTTTTGATGACCTGCTGCGACGCT
TTTTTTCTGGCAAGATAGTCTTGTAAATGCGGGCCAAGATCTGCACACTGGTATTTCGGTTTTTGGGG
CCGCGGGCGGCGACGGGGCCCGTGCGTCCCAGCGCACATGTTCGGCGAGGCGGGGCCTGCGAGCGCGG
CCACCGAGAATCGGACGGGGGTAGTCTCAAGCTGGCCGGCCTGCTCTGGTGCCTGGCCTCGCGCCGCC
GTGTATCGCCCCGCCCTGGGCGGCAAGGCTGGCCCGGTCGGCACCAGTTGCGTGAGCGGAAAGATGGC
CGCTTCCCGGCCCTGCTGCAGGGAGCTCAAAATGGAGGACGCGGCGCTCGGGAGAGCGGGCGGGTGAG
TCACCCACACAAAGGAAAAGGGCCTTTCCGTCCTCAGCCGTCGCTTCATGTGACTCCACTGAGTACCG
GGCGCCGTCCAGGCACCTCGATTAGTTCTCGTGCTTTTGGAGTACGTCGTCTTTAGGTTGGGGGGAGG
GGTTTTATGCGATGGAGTTTCCCCACACTGAGTGGGTGGAGACTGAAGTTAGGCCAGCTTGGCACTTG
ATGTAATTCTCCTTGGAATTTGCCCTTTTTGAGTTTGGATCTTGGTTCATTCTCAAGCCTCAGACAGT
GGTTCAAAGTTTTTTTCTTCCATTTCAGGTGTCGTGAGCTAGCCGCCACCATGGAGACCCTGCTGGGC
CTGCTGATCCTGTGGCTGCAGCTCCAGTGGGTGTCCAGCAAGCAGGAGGTGACCCAGATCCCTGCCGC
CCTGAGCGTGCCCGAGGGCGAGAACCTGGTGCTGAACTGCAGCTTCACCGACTCCGCCATCTACAACC
TGCAGTGGTTCCGGCAGGACCCCGGCAAGGGCCTGACCAGCCTGCTGCTGATCCAGAGCAGCCAGCGG
GAGCAGACCAGCGGACGGCTGAACGCCAGCCTGGACAAGAGCAGCGGCCGGAGCACCCTGTACATCGC
CGCCAGCCAGCCCGGCGACAGCGCCACCTACCTGTGCGCTGTGCGGCCTCTGTACGGCGGCAGCTACA
TCCCCACCTTCGGCAGAGGCACCAGCCTGATCGTGCACCCCTACATCCAGAACCCCGACCCCGCCGTG
TACCAGCTGCGGGACAGCAAGAGCAGCGACAAGTCTGTGTGCCTGTTCACCGACTTCGACAGCCAGAC
CAATGTGAGCCAGAGCAAGGACAGCGACGTGTACATCACCGACAAGACCGTGCTGGACATGCGGAGCA
TGGACTTCAAGAGCAACAGCGCCGTGGCCTGGAGCAACAAGAGCGACTTCGCCTGCGCCAACGCCTTC
AACAACAGCATTATCCCCGAGGACACCTTCTTCCCCAGCCCCGAGAGCAGCTGCGACGTGAAACTGGT
GGAGAAGAGCTTCGAGACCGACACCAACCTGAACTTCCAGAACCTGAGCGTGATCGGCTTCAGAATCC
TGCTGCTGAAGGTGGCCGGATTCAACCTGCTGATGACCCTGCGGCTGTGGAGCAGCGGCTCCCGGGCC
AAGAGAAGCGGATCCGGCGCCACCAACTTCAGCCTGCTGAAGCAGGCCGGAGACGTGGAAGAAAACCC
TGGCCCTAGGATGAGCATCGGCCTGCTGTGCTGCGCCGCCCTGAGCCTGCTGTGGGCAGGACCCGTGA
ACGCCGGAGTGACCCAGACCCCCAAGTTCCAGGTGCTGAAAACCGGCCAGAGCATGACCCTGCAGTGC
GCCCAGGACATGAACCACGAGTACATGAGCTGGTATCGGCAGGACCCCGGCATGGGCCTGCGGCTGAT
CCACTACTCTGTGGGAGCCGGAATCACCGACCAGGGCGAGGTGCCCAACGGCTACAATGTGAGCCGGA
GCACCACCGAGGACTTCCCCCTGCGGCTGCTGAGCGCTGCCCCCAGCCAGACCAGCGTGTACTTCTGC
GCCAGCAGCTATGTGGGCAACACCGGCGAGCTGTTCTTCGGCGAGGGCTCCAGGCTGACCGTGCTGGA
GGACCTGAAGAACGTGTTCCCCCCCGAGGTGGCCGTGTTCGAGCCCAGCGAGGCCGAGATCAGCCACA
CCCAGAAGGCCACACTGGTGTGTCTGGCCACCGGCTTCTACCCCGACCACGTGGAGCTGTCCTGGTGG
GTGAACGGCAAGGAGGTGCACAGCGGCGTGTCTACCGACCCCCAGCCCCTGAAGGAGCAGCCCGCCCT
GAACGACAGCCGGTACTGCCTGTCCTCCAGACTGAGAGTGAGCGCCACCTTCTGGCAGAACCCCCGGA
ACCACTTCCGGTGCCAGGTGCAGTTCTACGGCCTGAGCGAGAACGACGAGTGGACCCAGGACCGGGCC
AAGCCCGTGACCCAGATTGTGAGCGCCGAGGCCTGGGGCAGGGCCGACTGCGGCTTCACCAGCGAGAG
CTACCAGCAGGGCGTGCTGAGCGCCACCATCCTGTACGAGATCCTGCTGGGCAAGGCCACCCTGTACG
CCGTGCTGGTGTCTGCCCTGGTGCTGATGGCTATGGTGAAGCGGAAGGACAGCCGGGGCTAAGTCGAC
SEQ ID NO:7 Sense strand 5′ to 3′ sequence spanning the region between the MluI and SalI
restriction sites.
GCTAGCGCCGCCACCATGTTCCACGTGTCCTTCCGGTACATCTTCGGCCTGCCCCCCCTGATCCTGGT
GCTGCTGCCTGTGGCCAGCAGCGACTGCGACATCGAGGGCAAGGACGGCAAGCAGTACGAGAGCGTGC
TGATGGTGTCCATCGACCAGCTGCTGGACAGCATGAAGGAAATCGGCAGCAACTGCCTGAACAACGAG
TTCAACTTCTTCAAGCGGCACATCTGCGACGCCAACAAAGAAGGCATGTTCCTGTTCAGAGCCGCCAG
AAAGCTGCGGCAGTTCCTGAAGATGAACAGCACCGGCGACTTCGACCTGCATCTGCTGAAAGTGTCCG
AGGGCACCACCATCCTGCTGAATTGCACCGGCCAAGTGAAGGGCAGAAAGCCTGCCGCCCTGGGAGAA
GCCCAGCCTACCAAGAGCCTGGAAGAGAACAAGTCCCTGAAAGAGCAGAAGAAACTGAACGACCTGTG
CTTCCTGAAGCGGCTGCTGCAGGAAATCAAGACCTGCTGGAACAAGATCCTGATGGGCACCAAAGAGC
ACGGAAGCAGAGCCAAGAGAAGCGGCTCTGGCGAGGGCAGAGGCAGCCTGCTGACATGTGGCGACGTG
GAAGAAAACCCTGGCCCTATGGAGACCCTGCTGGGCCTGCTGATCCTGTGGCTGCAGCTCCAGTGGGT
GTCCAGCAAGCAGGAGGTGACCCAGATCCCTGCCGCCCTGAGCGTGCCCGAGGGCGAGAACCTGGTGC
TGAACTGCAGCTTCACCGACTCCGCCATCTACAACCTGCAGTGGTTCCGGCAGGACCCCGGCAAGGGC
CTGACCAGCCTGCTGCTGATCCAGAGCAGCCAGCGGGAGCAGACCAGCGGACGGCTGAACGCCAGCCT
GGACAAGAGCAGCGGCCGGAGCACCCTGTACATCGCCGCCAGCCAGCCCGGCGACAGCGCCACCTACC
TGTGCGCTGTGCGGCCTCTGTACGGCGGCAGCTACATCCCCACCTTCGGCAGAGGCACCAGCCTGATC
GTGCACCCCTACATCCAGAACCCCGACCCCGCCGTGTACCAGCTGCGGGACAGCAAGAGCAGCGACAA
GTCTGTGTGCCTGTTCACCGACTTCGACAGCCAGACCAATGTGAGCCAGAGCAAGGACAGCGACGTGT
ACATCACCGACAAGACCGTGCTGGACATGCGGAGCATGGACTTCAAGAGCAACAGCGCCGTGGCCTGG
AGCAACAAGAGCGACTTCGCCTGCGCCAACGCCTTCAACAACAGCATTATCCCCGAGGACACCTTCTT
CCCCAGCCCCGAGAGCAGCTGCGACGTGAAACTGGTGGAGAAGAGCTTCGAGACCGACACCAACCTGA
ACTTCCAGAACCTGAGCGTGATCGGCTTCAGAATCCTGCTGCTGAAGGTGGCCGGATTCAACCTGCTG
ATGACCCTGCGGCTGTGGAGCAGCGGCTCCCGGGCCAAGAGAAGCGGATCCGGCGCCACCAACTTCAG
CCTGCTGAAGCAGGCCGGAGACGTGGAAGAAAACCCTGGCCCTAGGATGAGCATCGGCCTGCTGTGCT
GCGCCGCCCTGAGCCTGCTGTGGGCAGGACCCGTGAACGCCGGAGTGACCCAGACCCCCAAGTTCCAG
GTGCTGAAAACCGGCCAGAGCATGACCCTGCAGTGCGCCCAGGACATGAACCACGAGTACATGAGCTG
GTATCGGCAGGACCCCGGCATGGGCCTGCGGCTGATCCACTACTCTGTGGGAGCCGGAATCACCGACC
AGGGCGAGGTGCCCAACGGCTACAATGTGAGCCGGAGCACCACCGAGGACTTCCCCCTGCGGCTGCTG
AGCGCTGCCCCCAGCCAGACCAGCGTGTACTTCTGCGCCAGCAGCTATGTGGGCAACACCGGCGAGCT
GTTCTTCGGCGAGGGCTCCAGGCTGACCGTGCTGGAGGACCTGAAGAACGTGTTCCCCCCCGAGGTGG
CCGTGTTCGAGCCCAGCGAGGCCGAGATCAGCCACACCCAGAAGGCCACACTGGTGTGTCTGGCCACC
GGCTTCTACCCCGACCACGTGGAGCTGTCCTGGTGGGTGAACGGCAAGGAGGTGCACAGCGGCGTGTC
TACCGACCCCCAGCCCCTGAAGGAGCAGCCCGCCCTGAACGACAGCCGGTACTGCCTGTCCTCCAGAC
TGAGAGTGAGCGCCACCTTCTGGCAGAACCCCCGGAACCACTTCCGGTGCCAGGTGCAGTTCTACGGC
CTGAGCGAGAACGACGAGTGGACCCAGGACCGGGCCAAGCCCGTGACCCAGATTGTGAGCGCCGAGGC
CTGGGGCAGGGCCGACTGCGGCTTCACCAGCGAGAGCTACCAGCAGGGCGTGCTGAGCGCCACCATCC
TGTACGAGATCCTGCTGGGCAAGGCCACCCTGTACGCCGTGCTGGTGTCTGCCCTGGTGCTGATGGCT
ATGGTGAAGCGGAAGGACAGCCGGGGCTAAGTCGAC
SEQ ID NO: 8 Nucleotide sequence of Constitutive-IL-7_T2A_TCR1. This sequence covers the
region between the NheI and SalI restriction sites.
MFHVSFRYIFGLPPLILVLLPVASSDCDIEGKDGKQYESVLMVSIDQLLDSMKEIGSNCLNNEFNFFK
RHICDANKEGMFLFRAARKLRQFLKMNSTGDFDLHLLKVSEGTTILLNCTGQVKGRKPAALGEAQPTK
SLEENKSLKEQFKLNDLCFLKRLLQEIKTCWNKILMGTKEHGSRAKRSGSGEGRGSLLTCGDVEENPG
PMETLLGLLILWLQLQWVSSKQEVTQIPAALSVPEGENLVLNCSFTDSAIYNLQWFRQDPGKGLTSLL
LIQSSQREQTSGRLNASLDKSSGRSTLYIAASQPGDSATYLCAVRPLYGGSYIPTFGRGTSLIVHPYI
QNPDPAVYQLRDSKSSDKSVCLFTDFDSQTNVSQSKDSDVYIIDKTVLDMRSMDFKSNSAVAWSNKSD
FACANAFNNSIIPEDTFFPSPESSCDVKLVEKSFETDTNLNFQNLSVIGFRILLLKVAGFNLLMTLRL
WSSGSRAKRSGSGATNFSLLKQAGDVEENPGPRMSIGLLCCAALSLLWAGPVNAGVTQTPKFQVLKTG
QSMTLQCAQDMNHEYMSWYRQDPGMGLRLIHYSVGAGITDQGEVPNGYNVSRSTTEDFPLRLLSAAPS
QTSVYFCASSYVGNTGELFFGEGSRLTVLEDLKNVFPPEVAVFEPSEAEISHTQKATLVCLATGFYPD
HVELSWWVNGKEVHSGVSTDPQPLKEQPALNDSRYCLSSRLRVSATFWQNPRNHFRCQVQFYGLDEND
EWTQDRAKPVTQIVSAEAWGRADCGFTSESYQQGVLSATILYEILLGKATLYAVLVSALVLMAMVKRK
DGRG
SEQ ID NO: 9 Protein sequence of constitutive IL-7_TCR1 IL7 is underlined, TCR is
italicised.
ACGCGTTAAGATACATTGATGAGTTTGGACAAACCACAACTAGAATGCAGTGAAAAAAATGCTTTATT
TGTGAAATTTGTGATGCTATTGCTTTATTTGTAACCATTATAAGCTGCAATAAACAAGTTCTCGAGTC
AGTGCTCTTTGGTGCCCATCAGGATCTTGTTCCAGCAGGTCTTGATTTCCTGCAGCAGCCGCTTCAGG
AAGCACAGGTCGTTCAGTTTCTTCTGCTCTTTCAGGGACTTGTTCTCTTCCAGGCTCTTGGTAGGCTG
GGCTTCTCCCAGGGCGGCAGGCTTTCTGCCCTTCACTTGGCCGGTGCAATTCAGCAGGATGGTGGTGC
CCTCGGACACTTTCAGCAGATGCAGGTCGAAGTCGCCGGTGCTGTTCATCTTCAGGAACTGCCGCAGC
TTTCTGGCGGCTCTGAACAGGAACATGCCTTCTTTGTTGGCGTCGCAGATGTGCCGCTTGAAGAAGTT
GAACTCGTTGTTCAGGCAGTTGCTGCCGATTTCCTTCATGCTGTCCAGCAGCTGGTCGATGGACACCA
TCAGCACGCTCTCGTACTGCTTGCCGTCCTTGCCCTCGATGTCGCAGTCGCTGCTGGCCACAGGCAGC
AGCACCAGGATCAGGGGGGGCAGGCCGAAGATGTACCGGAAGGACACGTGGAACATGGTGGCGGCCAT
ATGGATCCAACGAATGTCGAGAGGCTGGATCGGTCCCGGTGTCTTCTATGGAGGTCAAAACAGCGTGG
ATGGCGTCTCCAGGCGATCTGACGGTTCACTAAACGAGCTCTGCTTATATAGGCCTCCCACCGTACAC
GCCTAGACGTCACGCCTTCTGTATGAAACAGTTTTTCCTCCACGCCTTCTGTATGAAACAGTTTTTCC
TCCACGCCTTCTGTATGAAACAGTTTTTCCTCCGCTAGCGAGCTCAGGTACCGGCCAGTTAGGCCGAT
ATCATGCATCGTGAGGCTCCGGTGCCCGTCAGTGGGCAGAGCGCACATCGCCCACAGTCCCCGAGAAG
TTGGGGGGAGGGGTCGGCAATTGAACCGGTGCCTAGAGAAGGTGGCGCGGGGTAAACTGGGAAAGTGA
TGTCGTGTACTGGCTCCGCCTTTTTCCCGAGGGTGGGGGAGAACCGTATATAAGTGCAGTAGTCGCCG
TGAACGTTCTTTTTCGCAACGGGTTTGCCGCCAGAACACAGGTAAGTGCCGTGTGTGGTTCCCGCGGG
CCTGGCCTCTTTACGGGTTATGGCCCTTGCGTGCCTTGAATTACTTCCACCTGGCTGCAGTACGTGAT
TCTTGATCCCGAGCTTCGGGTTGGAAGTGGGTGGGAGAGTTCGAGGCCTTGCGCTTAAGGAGCCCCTT
CGCCTCGTGCTTGAGTTGAGGCCTGGCCTGGGCGCTGGGGCCGCCGCGTGCGAATCTGGTGGCACCTT
CGCGCCTGTCTCGCTGCTTTCGATAAGTCTCTAGCCATTTAAAATTTTTGATGACCTGCTGCGACGCT
TTTTTTCTGGCAAGATAGTCTTGTAAATGCGGGCCAAGATCTGCACACTGGTATTTCGGTTTTTGGGG
CCGCGGGCGGCGACGGGGCCCGTGCGTCCCAGCGCACATGTTCGGCGAGGCGGGGCCTGCGAGCGCGG
CCACCGAGAATCGGACGGGGGTAGTCTCAAGCTGGCCGGCCTGCTCTGGTGCCTGGCCTCGCGCCGCC
GTGTATCGCCCCGCCCTGGGCGGCAAGGCTGGCCCGGTCGGCACCAGTTGCGTGAGCGGAAAGATGGC
CGCTTCCCGGCCCTGCTGCAGGGAGCTCAAAATGGAGGACGCGGCGCTCGGGAGAGCGGGCGGGTGAG
TCACCCACACAAAGGAAAAGGGCCTTTCCGTCCTCAGCCGTCGCTTCATGTGACTCCACTGAGTACCG
GGCGCCGTCCAGGCACCTCGATTAGTTCTCGTGCTTTTGGAGTACGTCGTCTTTAGGTTGGGGGGAGG
GGTTTTATGCGATGGAGTTTCCCCACACTGAGTGGGTGGAGACTGAAGTTAGGCCAGCTTGGCACTTG
ATGTAATTCTCCTTGGAATTTGCCCTTTTTGAGTTTGGATCTTGGTTCATTCTCAAGCCTCAGACAGT
GGTTCAAAGTTTTTTTCTTCCATTTCAGGTGTCGTGAGCTAGCCGCCACCATGAAGAAGCACCTGACC
ACCTTTCTCGTGATCCTGTGGCTGTACTTCTACCGGGGCAACGGCAAGAACCAGGTGGAACAGAGCCC
CCAGAGCCTGATCATCCTGGAAGGCAAGAACTGCACCCTGCAGTGCAACTACACCGTGTCCCCCTTCA
GCAACCTGCGGTGGTACAAGCAGGACACCGGCAGAGGCCCTGTGTCCCTGACCATCCTGACCTTCAGC
GAGAACACCAAGAGCAACGGCCGGTACACCGCCACCCTGGACGCCGATACAAAGCAGAGCAGCCTGCA
CATCACCGCCAGCCAGCTGAGCGATAGCGCCAGCTACATCTGCGTGGTGTCCGGCGGCACAGACAGCT
GGGGCAAGCTGCAGTTTGGCGCCGGAACACAGGTGGTCGTGACCCCCGACATCCAGAACCCTGACCCT
GCCGTGTACCAGCTGCGGGACAGCAAGAGCAGCGACAAGAGCGTGTGCCTGTTCACCGACTTCGACAG
CCAGACCAACGTGTCCCAGAGCAAGGACAGCGACGTGTACATCACCGACAAGACCGTGCTGGACATGC
GGAGCATGGACTTCAAGAGCAATAGCGCCGTGGCCTGGTCCAACAAGAGCGACTTCGCCTGCGCCAAC
GCCTTCAACAACAGCATTATCCCCGAGGACACATTCTTCCCAAGCCCCGAGAGCAGCTGCGACGTCAA
GCTGGTGGAAAAGAGCTTCGAGACAGACACCAACCTGAACTTCCAGAACCTGAGCGTGATCGGCTTCA
GAATCCTGCTGCTGAAGGTGGCCGGCTTCAACCTGCTGATGACCCTGAGACTGTGGTCCAGCGGCAGC
CGGGCCAAGAGATCTGGATCCGGCGCTACCAACTTTAGCCTGCTGAAGCAGGCCGGGGACGTGGAAGA
AAACCCTGGCCCTAGGATGGCCAGCCTGCTTGTCTTCTGCGGCGCCTTCTACCTGCTGGGCACCGGCT
CTATGGATGCCGACGTGACCCAGACCCCCCGGAACAGAATCACCAAGACCGGCAAGCGGATCATGCTG
GAATGCTCCCAGACCAAGGGCCACGACCGGATGTACTGGTACAGACAGGACCCTGGCCTGGGCCTGCG
GCTGATCTACTACAGCTTCGACGTGAAGGACATCAACAAGGGCGAGATCAGCGACGGCTACAGCGTGT
CCAGACAGGCTCAGGCCAAGTTCAGCCTGTCCCTGGAAAGCGCCATCCCCAACCAGACCGCCCTGTAC
TTTTGTGCCACAAGCGGCCAGGGCGCCTACGAGGAGCAGTTCTTTGGCCCTGGCACCCGGCTGACAGT
GCTGGAAGATCTGAAGAACGTGTTCCCCCCAGAGGTGGCCGTGTTCGAGCCTTCTGAGGCCGAAATCA
GCCACACCCAGAAAGCCACACTCGTGTGTCTGGCCACCGGCTTCTACCCCGACCACGTGGAACTGTCT
TGGTGGGTCAACGGCAAAGAGGTGCACAGCGGCGTGTCCACCGATCCCCAGCCTCTGAAAGAACAGCC
CGCCCTGAACGACAGCCGGTACTGCCTGAGCAGCAGACTGAGAGTGTCCGCCACCTTCTGGCAGAACC
CCAGAAACCACTTCAGATGCCAGGTGCAGTTTTACGGCCTGAGCGAGAACGACGAGTGGACCCAGGAC
AGAGCCAAGCCCGTGACACAGATCGTGTCTGCCGAAGCTTGGGGGCGCGCCGATTGTGGCTTTACCAG
CGAGAGCTACCAGCAGGGCGTGCTGAGCGCCACCATCCTGTACGAGATCCTGCTGGGAAAGGCCACAC
TGTACGCCGTGCTGGTGTCTGCCCTGGTGCTGATGGCCATGGTCAAGCGGAAGGACAGCCGGGGCTAA
TAAGTCGAC
SEQ ID NO: 10 Sense strand 5′ to 3′ sequence spanning the region between the MluI and SalI
restriction sites (see FIG. 6).
MKKHLTIFLVILWLYFYRGNGKNQVEQSPQSLIILEGKNCTLQCNYIVSPFSNLRWYKQDTGRGPVSL
TILTFSENTKSNGRYTATLDADTKQSSLHITASQLSDSASYICVVSGGIDSWGKLQFGASTQVVVIPD
IQNPDPAVYQLRDSKSSDKSVCLFTDFDSQINVSQSKDSDVYIIDKTVLDMRSMDFKSMSAVAWSNKS
DFACANAFNNSIIPEDIFFPSPESSCDVKLVEKSFEIDTNLNFQNLSVIGFRILLLKVAGFNLLMTLR
LWSSGSRAKRSGSGATNFSLLKQAGDVEENPGPRMASLLFFCGAFYLLGTGSMDADVTQIPRNRITKT
GKRIMLECSQTKGHDRMYWYRQDPGLGLRLTYYSFDVKDINKGEISDGYSVSRQAQAKFSLSLESAIP
NQTALYFCATSGQGAYEEQFFGPGTRLTVLEDLKNVFPPEVAVFEPSEAEISHTQKATLVCLAIGFYP
DHVELSWWVNGKEVHSGVSIDPQPLKEQPALNDSRYCLSSRLRVSATFWQNFRNHFRCQVQFYGLESN
DEWTQDRAKPVTQIVSAEAWGRADCGFTSESYQQGVLSATILYEILLGKATLYAVLVSALVLMAMVKR
KDSRG
SEQ ID NO: 11 TCR2 sequence
GCTAGCGCCGCCACCATGTTCCACGTGTCCTTCCGGTACATCTTCGGCCTGCCCCCCCTGATCCTGGT
GCTGCTGCCTGTGGCCAGCAGCGACTGCGACATCGAGGGCAAGGACGGCAAGCAGTACGAGAGCGTGC
TGATGGTGTCCATCGACCAGCTGCTGGACAGCATGAAGGAAATCGGCAGCAACTGCCTGAACAACGAG
TTCAACTTCTTCAAGCGGCACATCTGCGACGCCAACAAAGAAGGCATGTTCCTGTTCAGAGCCGCCAG
AAAGCTGCGGCAGTTCCTGAAGATGAACAGCACCGGCGACTTCGACCTGCATCTGCTGAAAGTGTCCG
AGGGCACCACCATCCTGCTGAATTGCACCGGCCAAGTGAAGGGCAGAAAGCCTGCCGCCCTGGGAGAA
GCCCAGCCTACCAAGAGCCTGGAAGAGAACAAGTCCCTGAAAGAGCAGAAGAAACTGAACGACCTGTG
CTTCCTGAAGCGGCTGCTGCAGGAAATCAAGACCTGCTGGAACAAGATCCTGATGGGCACCAAAGAGC
ACGGAAGCAGAGCCAAGAGAAGCGGCTCTGGCGCGCCCGTGAAGCAGACCCTGAACTTCGACCTGCTG
AAACTGGCCGGCGACGTGGAAAGCAACCCTGGCCCTATGAAGAAGCACCTGACCACCTTTCTCGTGAT
CCTGTGGCTGTACTTCTACCGGGGCAACGGCAAGAACCAGGTGGAACAGAGCCCCCAGAGCCTGATCA
TCCTGGAAGGCAAGAACTGCACTCTGCAGTGCAACTACACCGTGTCCCCCTTCAGCAACCTGCGCTGG
TACAAGCAGGATACCGGCAGAGGCCCTGTGTCCCTGACCATCCTGACCTTCAGCGAGAACACCAAGAG
CAACGGCCGGTACACCGCCACCCTGGACGCCGATACAAAGCAGAGCAGCCTGCACATCACCGCCTCCC
AGCTGAGCGATAGCGCCAGCTACATCTGCGTGGTGTCCGGCGGCACAGACAGCTGGGGCAAGCTGCAG
TTTGGCGCCGGAACACAGGTGGTCGTGACCCCCGACATCCAGAACCCTGACCCTGCCGTGTACCAGCT
GCGGGACAGCAAGAGCAGCGACAAGAGCGTGTGCCTGTTCACCGACTTCGACTCCCAGACCAACGTGT
CCCAGAGCAAGGACAGCGACGTGTACATCACCGACAAGACCGTGCTGGATATGCGGAGCATGGACTTC
AAGAGCAATAGCGCCGTGGCCTGGTCTAACAAGAGCGACTTCGCCTGCGCCAACGCCTTCAACAACAG
CATTATCCCCGAGGACACATTCTTCCCAAGCCCCGAGAGCAGCTGCGACGTGAAACTGGTGGAAAAGA
GCTTCGAGACAGACACCAACCTGAATTTCCAGAACCTGAGCGTGATCGGCTTCCGGATCCTGCTGCTG
AAGGTGGCCGGATTCAACCTGCTGATGACCCTGCGGCTGTGGTCCTCTGGCTCTCGGGCCAAGAGAAG
CGGCAGCGGCGCCACCAATTTCAGCCTGCTGAAGCAGGCAGGGGATGTGGAAGAGAATCCCGGCCCTA
GAATGGCCTCCCTGCTGTTTTTCTGCGGCGCCTTCTACCTGCTGGGGACCGGCAGCATGGACGCTGAC
GTGACCCAGACCCCCCGGAACAGAATCACCAAGACCGGCAAGCGGATCATGCTGGAATGCAGCCAGAC
AAAGGGCCACGACCGGATGTACTGGTACAGACAGGATCCAGGACTGGGCCTGAGGCTGATCTACTACA
GCTTCGATGTGAAGGACATCAACAAGGGCGAGATCAGCGACGGCTACAGCGTGTCCAGACAGGCCCAG
GCCAAGTTCTCCCTGAGCCTGGAAAGCGCCATCCCCAACCAGACCGCCCTGTACTTTTGTGCCACAAG
CGGCCAGGGCGCCTACGAGGAACAGTTCTTTGGCCCTGGCACCCGGCTGACAGTGCTGGAAGATCTGA
AGAACGTGTTCCCCCCAGAGGTGGCAGTGTTCGAGCCTAGCGAGGCCGAGATCTCCCACACCCAGAAA
GCCACACTCGTGTGTCTGGCCACCGGATTCTACCCCGACCATGTGGAACTGTCTTGGTGGGTCAACGG
CAAAGAGGTGCACAGCGGCGTGTCCACCGATCCCCAGCCTCTGAAAGAACAGCCCGCCCTGAACGACA
GCCGGTACTGCCTGAGCAGCAGACTGAGAGTGTCCGCCACCTTCTGGCAGAACCCCAGAAATCACTTC
AGATGCCAGGTGCAGTTTTACGGCCTGAGCGAGAACGACGAGTGGACCCAGGATAGGGCCAAGCCCGT
GACTCAGATCGTGTCTGCCGAAGCCTGGGGCAGAGCCGATTGCGGCTTTACCAGCGAGAGCTACCAGC
AGGGCGTGCTGAGCGCCACCATCCTGTACGAGATCCTGCTGGGCAAGGCCACACTGTACGCCGTGCTG
GTGTCTGCCCTGGTGCTGATGGCCATGGTCAAGCGGAAGGACAGCCGGGGCTGATGAGGTCGAC
SEQ ID NO: 12 Nucleotide sequence covering the region between the NheI and SalI restriction
sites.
MFHVSFRYIFGLPPLILVLLPVASSDCDIEGKDGKQYESVLMVSIDQLLDSMKEIGSNCLNNEFNFFK
RHICDANKEGMFLFRAARKLRQFLKMNSTGDFDLHLLKVSEGTTILLNCTGQVKGRKPAALGEAQPTK
SLEENKSLKEQFKLNDLCFLKRLLQEIKTCWNKILMGTKEHGSRAKRSGSGAPVKQTLNFDLLKLAGD
VESNPGPMKKHLTTFLVILWLYFYRGNGKNQVEQSPQSLIILEGKNCTLQCNYTVSPFSNLRWYKQDT
GRGPVSLTILTFSENTKSNGRYTATLDADTKQSSLHITASQLSDSASYICVVSGGTDSWGKLQFGAGT
QVVVTPDIQNPDPAVYQLRDSKSSDKSVCLFTDFDSQTNVSQSKDSDVYITDKTVLDMRSMDFKSNSA
VAWSNKSDFACANAFNNSIIPEDTFFPSPESSCDVKLVEKSFETDTNLNFQNLSVIGFRILLLKVAGF
NLLMTLRLWSSGSRAKRSGSGATNFSLLKQAGDVEENPGPRMASLLFFCGAFYLLGTGSMDADVTQTP
RNRITKTGKRIMLECSQTKGHDRMYWYRQDPGLGLRLIYYSFDVKDINKGEISDGYSVSRQAQAKFSL
SLESAIPNQTALYFCATSGQGAYEEQFFGPGTRLTVLEDLKNVFPPEVAVFEPSEAEISHTQKATLVC
LATGFYPDHVELSWWVNGKEVHSGVSTDPQPLKEQPALNDSRYCLSSRLRVSATFWQNPRNHFRCQVQ
FYGLSENDEWTQDRAKPVTQIVSAEAWGRADCGFTSESYQQGVLSATILYEILLGKATLYAVLVSALV
LMAMVKRKDSRG**
SEQ ID NO: 13 Translated protein sequence of constitutive IL-7_TCR2
GGAGGAAAAACTGTTTCATACAGAAGGCGT
SEQ ID NO: 14 human IL2 promoter NFAT TRE
TAGGCGTGTACGGTGGGAGGCCTATATAAGCAGAGCTCGTTTAGTGAACCGTCAGATCGCCTGGAGAC
GCCATCCACGCTGTTTTGACCTCCATAGAAGACACCGGGACCGATCCAGCCTCTCGACATTCGTTGGA
TC
SEQ ID NO: 15 minimal CMV promoter
```

1. A T cell comprising a constitutively expressed antigen receptor and an inducibly expressed IL-7 molecule, wherein the antigen receptor is a chimeric antigen receptor (CAR) that binds to a tumor antigen, or is a T cell receptor (TCR) that binds to an MHC-displayed peptide fragment of the tumor antigen.

2. The T cell according to claim 1 wherein the T cell comprises
i. a first nucleotide sequence encoding the IL-7 molecule,
ii. a second nucleotide sequence encoding the antigen receptor,
iii. an inducible promoter operably linked to the first nucleotide sequence; and
iv. a constitutive promoter operably linked to the second nucleotide.

3. The T cell according to claim 1, wherein expression of the IL-7 molecule is induced by the activation of the T cell.

4. The T cell according to claim 1, wherein expression of the IL-7 molecule is from the inducible promoter Nuclear Factor of Activated T cells (NFAT) Transcriptional Response Element (TRE).

5. The T cell according to claim 4, comprising a nucleic acid sequence encoding the NFAT TRE, wherein the nucleic acid sequence encoding the NFAT TRE comprises SEQ ID NO: 14 or a variant thereof.

6. The T cell according to claim 4, wherein the inducible promoter comprises three or more copies of the NFAT TRE.

7. The T cell according to claim 1, wherein expression of the antigen receptor is from the constitutive promoter human elongation factor-1 alpha (EF1A).

8. The T cell according to claim 1, wherein the IL-7 is human IL-7.

9. The T cell according to claim 1, wherein the antigen receptor is a TCR.

10. The T cell according to claim 9, wherein the TCR is an affinity enhanced TCR.

11. The T cell according to claim 9, wherein the TCR comprises the amino acid sequence of any one of SEQ ID NOs: 5, 6 or 11.

12. The T cell according to claim 1, wherein the antigen receptor is a CAR.

13. The T cell according to claim 1, wherein the tumor antigen is NY-ESO1, PRAME, alpha-fetoprotein (AFP), MAGE A4, MAGE A1, MAGE A10 or MAGE B2.

14. A pharmaceutical composition comprising the T cell according to claim 1 and a pharmaceutically acceptable excipient.

15. A method of treating cancer comprising administering to a patient having cancer the T cell according to claim 1, wherein the cancer expresses the tumor antigen.

16. The method of claim 15, wherein the tumor antigen is NY-ESO1, PRAME, alpha-fetoprotein (AFP), MAGE A4, MAGE A1, MAGE A10 or MAGE B2.

17. A method of treating cancer comprising administering to a patient having cancer the pharmaceutical composition according to claim 14, wherein the cancer expresses the tumor antigen.

18. The method of claim 17, wherein the tumor antigen is NY-ESO1, PRAME, alpha-fetoprotein (AFP), MAGE A4, MAGE A1, MAGE A10 or MAGE B2.

19. A method of producing a population of modified T cells, the method comprising introducing into a population of T cells a nucleic acid construct comprising
i. a first nucleotide sequence encoding IL-7,
ii. a second nucleotide sequence encoding an antigen receptor, wherein the antigen receptor is a chimeric antigen receptor (CAR) that binds to a tumor antigen, or is a T cell receptor (TCR) that binds to an MHC-displayed peptide fragment of the tumor antigen;
iii. an inducible promoter operably linked to the first nucleotide sequence; and
iv. a constitutive promoter operably linked to the second nucleotide.
